(19) United States
(12) Patent Application Publication
(10) Pub. No.: US 2026/0209290 A1
(43) Pub. Date: Jul. 23, 2026

(54) METHODS, PHARMACEUTICAL COMPOSITIONS, AND PHARMACEUTICAL PREPARATIONS FOR PREVENTION AND/OR TREATMENT OF PULMONARY ARTERIAL HYPERTENSION

(71) Applicant: FUWAI HOSPITAL, CHINESE ACADEMY OF MEDICAL SCIENCES, BEIJING, CHINA, Beijing (CN)

(72) Inventors: Xiaojian WANG, Beijing (CN); Yuqi XIE, Beijing (CN); Weixian YANG, Beijing (CN); Liping YU, Beijing (CN); Chilu LIU, Beijing (CN); Xiaoyue HU, Beijing (CN)

(73) Assignee: FUWAI HOSPITAL, CHINESE ACADEMY OF MEDICAL SCIENCES, BEIJING, CHINA, Beijing (CN)

(21) Appl. No.: 19/639,203

(22) Filed: Apr. 4, 2026

Related U.S. Application Data
(63) Continuation-in-part of application No. PCT/CN2024/109886, filed on Aug. 5, 2024.

(30) Foreign Application Priority Data
Aug. 4, 2023 (CN) 202310978261.X

(51) Int. Cl. C07K 14/47; A61K 38/00; A61P 9/00; C12N 15/86

(52) U.S. Cl. CPC C07K 14/47; A61P 9/00; C12N 15/86; A61K 38/00; C12N 2710/10343

(57) ABSTRACT

Disclosed is a method, a pharmaceutical composition, and a pharmaceutical preparation for prevention and/or treatment of pulmonary arterial hypertension (PAH). The method includes administering to a subject at least one of a first agent that inhibits binding of Hic-5 to SMAD7 or a second agent that inhibits Hic-5.

### CROSS-REFERENCE TO RELATED APPLICATIONS

This application is a continuation-in-part application of International Application No. PCT/CN2024/109886, filed on Aug. 5, 2024, which claims priority to Chinese Patent Application No. 202310978261.X, filed on Aug. 4, 2023, and titled “Use of PLEKHH2 in Diagnosis and Treatment of Pulmonary Arterial Hypertension”, the entire contents of each of which are hereby incorporated by reference.

### SEQUENCE LISTING

This application contains a Sequence Listing which has been submitted electronically in XML format and is hereby incorporated by reference in its entirety. The XML copy, created on Apr. 3, 2026, is named “2026 Apr. 3-Sequence Listing-6B811-H016US00” and is 18,965 bytes in size.

### TECHNICAL FIELD

The present disclosure generally relates to the field of biomedicine, and in particular to, a method, a pharmaceutical composition, and a pharmaceutical preparation for prevention and/or treatment of pulmonary arterial hypertension.

### BACKGROUND

Pulmonary arterial hypertension (PAH) is a malignant pulmonary vascular disease characterized primarily by elevated pulmonary arterial pressure and progressive increase in pulmonary vascular resistance, which often leads to severe right heart failure and even death. Idiopathic pulmonary arterial hypertension (IPAH) and heritable pulmonary arterial hypertension (HPAH) are the most predominant types of PAH, accounting for 37%-49% of the first major category of PAH. IPAH and HPAH are rare diseases with an incidence of 5-10 per million population. IPAH and HPAH can occur in all age groups from children to adults. The most critical clinical challenge currently faced by IPAH and HPAH is the lack of truly effective therapeutic drugs. Existing PAH therapeutics are all “symptomatic treatments” that dilate pulmonary blood vessels, which can only delay disease progression, and no drug can effectively reverse the pathological remodeling of pulmonary blood vessels in patients. The substantial disease burden of IPAH and HPAH has attracted high-level attention from the state. In 2018, the National Health Commission and four other ministries included IPAH and HPAH in the “First List of Rare Diseases in China,” clearly identifying them as important national rare diseases. Therefore, it is urgent to further accelerate basic research related to PAH and discover new drug targets.

IPAH and HPAH are monogenic autosomal dominant genetic diseases with obvious familial inheritance and aggregation. As of December 2022, a total of 21 PAH-associated genes (among which two genes, BMP9 and PTGIS, were originally reported by the inventor's research group of the present application) have been identified worldwide, which can generally explain the etiology of 50%-70% of HPAH patients and 20%-40% of IPAH patients. PAH-associated genetic genes are not only a breakthrough for understanding the etiology of PAH patients, but also an important “key” to uncovering the pathological mechanism of PAH and developing new drugs. Most of the currently known PAH-causative genes for PAH (e.g., BMPR2, ALK1, ENG, CAV1, SOX17, and KDR) are highly expressed in pulmonary vascular endothelial cells, suggesting that pulmonary vascular endothelial cell injury is the initiating cause of PAH pathogenesis. Targeting PAH-causative genes such as BMPR2 and SOX17, upregulating the expression or enhancing the activity of these genes can protect pulmonary arterial endothelial cells (PAECs), inhibit proliferation, and reverse mesenchymal transition, demonstrating good preventive or therapeutic effects on PAH in animal models. Some drugs targeting these genes have entered clinical trials and represent a current global hotspot in PAH drug development.

Consequently, the identification of PAH-associated genes has dual clinical significance. On one hand, pathogenic mutations are the true cause of IPAH and HPAH, which are crucial for clinical diagnosis. On the other hand, if a causative gene is confirmed to have a protective function, the causative gene will become a new potential target, providing new possibilities for clinical treatment.

### SUMMARY

One or more embodiments of the present disclosure provide a method for prevention and/or treatment of PAH. The method comprises administering to a subject at least one of a first agent that inhibits binding of hydrogen peroxide-inducible clone 5 (Hic-5) to mothers against decapentaplegic homolog 7 (SMAD7) or a second agent that inhibits Hic-5.

In some embodiments, the first agent may comprise at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a clustered regularly interspaced short palindromic repeats (CRISPR) gene editing agent targeting a binding site between Hic-5 and SMAD7; and the second agent may comprise at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting Hic-5.

In some embodiments, the binding site between Hic-5 and SMAD7 may be a LIM3 domain of Hic-5.

In some embodiments, the first agent may competitively bind to a LIM3 domain of Hic-5 with SMAD7.

In some embodiments, the first agent may comprise at least one of PLEKHH2 or a PLEKHH2 activator.

In some embodiments, the at least one of PLEKHH2 or the PLEKHH2 activator may comprise at least one of a PLEKHH2 gene, a PLEKHH2 mRNA, a PLEKHH2 cDNA, a PLEKHH2 protein, a peptide fragment of the PLEKHH2 protein, an analog of the PLEKHH2 protein, an active fragment of any of the foregoing, a vector expressing PLEKHH2, nanoparticles carrying the PLEKHH2 gene, a viral vector carrying the PLEKHH2 gene, a PEGylated protein encapsulating the PLEKHH2 gene or the PLEKHH2 protein, protein microspheres encapsulating the PLEKHH2 gene or the PLEKHH2 protein, a liposome encapsulating the PLEKHH2 gene or the PLEKHH2 protein, or extracellular vesicles encapsulating the PLEKHH2 gene or the PLEKHH2 protein.

In some embodiments, the at least one of PLEKHH2 or the PLEKHH2 activator may prevent, treat, alleviate, and/or ameliorate the PAH by inhibiting hyperphosphorylation of SMAD2/3.

In some embodiments, the second agent may be selected from a group comprising an siRNA targeting an Hic-5 gene, an shRNA targeting the Hic-5 gene, an agent that inhibits transcriptional activity of the Hic-5 gene, an agent that inhibits a transcription level of an Hic-5 mRNA, an agent that promotes degradation of the Hic-5 mRNA, an agent that inhibits translation of the Hic-5 mRNA, an agent that specifically recognizes the Hic-5 gene with a guide nucleic acid and cleaves the Hic-5 gene to reduce an expression level of Hic-5, or a reagent for partial or complete knockout of the Hic-5 gene.

In some embodiments, the siRNA targeting the Hic-5 gene may have a sequence shown in SEQ ID NOs: 9-10.

In some embodiments, the second agent may be capable of reducing the PAH, increasing BMPR2 pathway activity, reducing TGF-β pathway activity, or restoring BMPR2-TGF-β pathway balance.

One or more embodiments of the present disclosure also provide a method for prevention and/or treatment of PAH. The method comprises administering to a subject at least one of PLEKHH2 or a PLEKHH2 activator.

One or more embodiments of the present disclosure also provide a pharmaceutical composition for prevention and/or treatment of PAH. The pharmaceutical composition comprises at least one of a first agent that inhibits binding of Hic-5 to SMAD7 or a second agent that inhibits Hic-5; wherein the first agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting a binding site between Hic-5 and SMAD7; and the second agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting Hic-5.

In some embodiments, the pharmaceutical composition may further comprise other drugs for prevention and/or treatment of PAH.

In some embodiments, the other drugs for prevention and/or treatment of PAH may comprise at least one of a calcium channel blocker, a prostacyclin analog, an endothelin receptor antagonist, a phosphodiesterase-5 inhibitor, or a guanylate cyclase stimulator.

In some embodiments, the pharmaceutical composition may further comprise at least one of a pharmaceutically acceptable carrier or an excipient.

One or more embodiments of the present disclosure also provide a pharmaceutical preparation for prevention and/or treatment of PAH. The pharmaceutical preparation comprises the pharmaceutical composition.

In some embodiments, a dosage form of the pharmaceutical preparation may comprise at least one of an injection, a lyophilized powder, a solution, a tablet, a capsule, a granule, an ointment, a cream, a gel, a suspension, an oral solution, a controlled-release formulation, a nanocrystal formulation, a microemulsion, or a solid dispersion.

One or more embodiments of the present disclosure also provide a method for screening a candidate drug for prevention and/or treatment of PAH. The method comprises: screening an agent that binds to a LIM3 domain of Hic-5 as the candidate drug; or simulating a binding site between Hic-5 and PLEKHH2, and screening an agent that binds to the binding site between Hic-5 and PLEKHH2 as the candidate drug; or simulating a binding site between Hic-5 and SMAD7, and screening an agent that binds to the binding site between Hic-5 and SMAD7 as the candidate drug; or treating a system expressing or containing a PLEKHH2 gene with a test substance; detecting an expression level of the PLEKHH2 gene in the system; and selecting a test substance that increases the expression level of the PLEKHH2 gene as the candidate drug.

One or more embodiments of the present disclosure also provide a method for treating PAH. The method comprises: detecting an expression level of PLEKHH2 in a sample from a subject; comparing the sample from the subject with a control sample; and administering an effective amount of at least one of PLEKHH2 or a PLEKHH2 activator to the subject in response to determining that the expression level of PLEKHH2 in the sample from the subject is lower than that in the control sample.

In some embodiments, the sample from the subject may comprise at least one of blood, tissues, blood-derived cells, serum, plasma, lymph, synovial fluid, or cells from the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a genetic pedigree chart illustrating that 6 families are found to carry known pathogenic mutations among 10 families with familial PAH according to some embodiments of the present disclosure;

FIG. 2 is a genetic pedigree chart illustrating identification of PLEKHH2 as a novel potential causative gene for PAH in two HPAH families according to some embodiments of the present disclosure, where W/W indicates wild type, and I1245T/W and S658F/W indicate heterozygous mutations of PLEKHH2;

FIG. 3 is a chart illustrating expression levels of PLEKHH2 gene in various tissues from the National Center for Biotechnology Information (NCBI) database according to some embodiments of the present disclosure, where panel A illustrates expression levels of PLEKHH2 in various adult tissues, and panel B illustrates expression levels of PLEKHH2 in various tissues during developmental stages;

FIG. 4 is a chart illustrating expression levels of the PLEKHH2 gene in a plurality of organ tissues of rats detected by Quantitative Real-time Polymerase Chain Reaction (qPCR) according to some embodiments of the present disclosure;

FIG. 5 is an immunofluorescence image illustrating primary localization of PLEKHH2 protein according to some embodiments of the present disclosure, where blue indicates 4′,6-diamidino-2-phenylindole (DAPI) nuclear staining, red indicates Von Willebrand Factor (VWF) marking endothelial cells, and green indicates PLEKHH2 protein;

FIG. 6 is a diagram illustrating PLEKHH2 protein expression levels in three types of cells: pulmonary artery smooth muscle cells (PASMCs), pulmonary microvascular endothelial cells (PMECs), and pulmonary artery endothelial cells (PAECs) detected by Western blot according to some embodiments of the present disclosure, where panel A illustrates a Western blot image, panel B illustrates a statistical chart of the PLEKHH2 protein expression levels, * indicates that P is less than 0.05, ** indicates that P is less than 0.01, and n=3;

FIG. 7 is a diagram illustrating expression levels of the PLEKHH2 gene in lung tissues of monocrotaline (MCT)-induced PAH rats according to some embodiments of the present disclosure, where panel A illustrates a statistical chart of a mean pulmonary arterial pressure (mPAP) for a wild-type control rat group (also referred to as a WT group) and a PAH rat model group (also referred to as a MCT group) constructed by subcutaneous injection of 60 mg/kg MCT, panel B illustrates a statistical chart of a right ventricular hypertrophy index (Right Ventricle (RV))/(Left Ventricle (LV)+Septum(S)) for the WT group and the MCT group, panel C illustrates mRNA expression levels of the PLEKHH2 gene in the WT group and the MCT group, panel D illustrates a Western blot image and a statistical chart, ** indicates that P is less than 0.01, *** indicates that P is less than 0.001, **** indicates that P is less than 0.0001 versus the WT group, and n=5;

FIG. 8 is a diagram illustrating expression levels of the PLEKHH2 gene in lung tissues of rats with hypoxia and Sugen-induced PAH according to some embodiments of the present disclosure, where panel A illustrates a statistical chart of mPAP for the WT group and a PAH rat model group (also referred to as a SuHx group, where SuHx refers to the Sugen 5416 plus hypoxia model) constructed by subcutaneous injection of Su-5416 (20 mg/kg) followed by 3 weeks of hypoxia and 2 weeks of reoxygenation, panel B illustrates a statistical chart of the right ventricular hypertrophy index (RV/(LV+S)) for the WT group and the SuHx group, panel C illustrates mRNA expression levels of the PLEKHH2 gene in the WT group and the SuHx group, panel D illustrates a Western blot image and a statistical chart, ** indicates that P is less than 0.01, *** indicates that P is less than 0.001, **** indicates that P is less than 0.0001 versus the WT group, and n=5;

FIG. 9 is an immunofluorescence image illustrating expression of the PLEKHH2 gene in endothelial cells of lung tissues from PAH patients according to some embodiments of the present disclosure, where Control indicates a control lung tissue without PAH, and PAH indicates a lung tissue from patients with congenital heart disease combined with severe PAH;

FIG. 10 is a diagram illustrating expression levels of the PLEKHH2 gene after transfection of the PAECs with siRNA-PLEKHH2 according to some embodiments of the present disclosure, where panel A illustrates a Western blot image, panel B illustrates a statistical chart of PLEKHH2 protein expression levels, * indicates that P is less than 0.05 versus a si-NC group, and n=3;

FIG. 11 is a diagram illustrating the effect of PLEKHH2 gene knockdown on a proliferative capacity of the PAECs detected by a 5-ethynyl-2′-deoxyuridine (EDU) cell proliferation assay according to some embodiments of the present disclosure, where Panel A illustrates EDU staining images; Panel B illustrates a statistical graph of EDU-positive cells in a Normo group; Panel C illustrates a statistical graph of EDU-positive cells in a Hypo group; the Normo group indicates normoxia (an oxygen concentration of 20% (v/v)); the Hypo group indicates hypoxia (an oxygen concentration of 3% (v/v)); * indicates that P is less than 0.05, ** indicates that P is less than 0.01, *** indicates that P is less than 0.001, **** indicates that Pis less than 0.0001 versus the si-NC group, and n=3;

FIG. 12 is a diagram illustrating the effect of PLEKHH2 gene knockdown on expression of Bcl-2 and Bax in PAECs under normoxia detected by Western blot according to some embodiments of the present disclosure, where Panel A illustrates a Western blot image of the Bcl-2; Panel B illustrates a statistical graph of Bcl-2 expression levels; Panel C illustrates a Western blot image of the Bax; Panel D illustrates a statistical graph of Bax expression levels; * indicates that P is less than 0.05, ** indicates that P is less than 0.01, *** indicates that P is less than 0.001, **** indicates that P is less than 0.0001 versus the si-NC group, and n=3;

FIG. 13 is an image illustrating the effect of PLEKHH2 gene knockdown on tube formation capacity of PAECs under normoxia or hypoxia detected by Matrigel angiogenesis assay according to some embodiments of the present disclosure, where the Normo group indicates normoxia (the oxygen concentration of 20% (v/v)); and the Hypo group indicates hypoxia (the oxygen concentration of 3% (v/v));

FIG. 14 is a diagram illustrating changes in the expression and phosphorylation of key proteins of BMPR2/TGF-β pathway in the PAECs after PLEKHH2 gene knockdown detected by Western blot according to some embodiments of the present disclosure, where Panel A illustrates a Western blot image; Panel B illustrates a statistical graph of PLEKHH2 expression levels; Panel C illustrates a statistical graph of BMPR2 expression levels; Panel D illustrates a statistical graph of p-SMAD1/5/8/SMAD1/5/8 expression levels; Panel E illustrates a statistical graph of SMAD1/5/8 expression levels; Panel F illustrates a statistical graph of p-SMAD2/3/SMAD2/3 expression levels; Panel G illustrates a statistical graph of inhibitor of DNA binding 1 (ID1) expression levels; * indicates that P is less than 0.05, ** indicates that P is less than 0.01 versus the si-NC group, and n is in a range of 1-3;

FIG. 15 is a diagram illustrating results of in vivo upregulation of the PLEKHH2 gene effectively preventing PAH according to some embodiments of the present disclosure, where Panel A illustrates a schematic diagram of an experimental design; Panels B-C illustrate the effect of PLEKHH2 overexpression on right ventricular systolic pressure (RVSP) in MCT-induced rats detected by right heart catheterization; and Panels D-H illustrate the effect of PLEKHH2 overexpression on a structure and function of the right ventricle in the MCT-induced rats detected by echocardiography, wherein PAT/PET indicates a pulmonary artery acceleration time/a pulmonary ejection time, RVOT indicates a right ventricular outflow tract width, TAPSE indicates a tricuspid annular plane systolic excursion, RVID indicates a right ventricular internal dimension, and RVAW indicates a right ventricular anterior wall thickness;

FIG. 16 is a diagram illustrating results of in vivo upregulation of the PLEKHH2 gene effectively treating PAH according to some embodiments of the present disclosure, where Panel A illustrates a schematic diagram of an experimental design; Panels B-C illustrate results the effect of PLEKHH2 treatment on the RVSP in the MCT-induced rats detected by right heart catheterization; and Panels D-H illustrate the effect of PLEKHH2 treatment on the structure and function of the right ventricle in the MCT-induced rats detected by echocardiography, wherein PAT/PET indicates a pulmonary artery acceleration time/a pulmonary ejection time, RVOT indicates a right ventricular outflow tract width, TAPSE indicates a tricuspid annular plane systolic excursion, RVID indicates a right ventricular internal dimension, and RVAW indicates a right ventricular anterior wall thickness;

FIG. 17 is a Western blot image illustrating PLEKHH2, Hic-5, and SMAD7 protein expression in the lung tissue of the MCT-induced rats according to some embodiments of the present disclosure, where NC indicates a control group subcutaneously injected with normal saline; MCT indicates a model group subcutaneously injected with 60 mg/kg MCT;

FIG. 18 is an immunofluorescence image of pulmonary artery endothelial cells according to some embodiments of the present disclosure, where blue indicates nucleus, green indicates Hic-5, and red indicates PLEKHH2;

FIG. 19 is a schematic diagram illustrating protein structures of PLEKHH2, Hic-5, and SMAD7 according to some embodiments of the present disclosure;

FIG. 20 is a diagram illustrating the binding ability and binding sites between Hic-5 and SMAD7 and between Hic-5 and PLEKHH2 predicted using AlphaFold-3 software according to some embodiments of the present disclosure, where in Panel A, green indicates MH2 and N-terminal domains of a SMAD7 protein, and blue indicates a full-length Hic-5 protein; in Panel B, green indicates the full-length Hic-5 protein, blue indicates a FIRM domain of PLEKHH2, and purple indicates a protein sequence of Hic-5 that directly binds to SMAD7;

FIG. 21 is a diagram illustrating the binding between PLEKHH2 and Hic-5, and between SMAD7 and Hic-5 detected by Co-immunoprecipitation (Co-IP) according to some embodiments of the present disclosure, where sh-P indicates PLEKHH2 gene knockdown using an sh-RNA; Ad-P indicates PLEKHH2 gene overexpression using an adenovirus; and Ad indicates an empty adenovirus vector as control;

FIG. 22 is a diagram illustrating the binding between PLEKHH2 and Hic-5, and between SMAD7 and Hic-5 in the lung tissue of rats after MCT stimulation detected by Co-IP according to some embodiments of the present disclosure, where WT indicates wild-type rats injected with normal saline as control; WT+MCT indicates lung tissue collected from wild-type rats three weeks after injection with MCT (40 mg/kg); and P-KO+MCT indicates lung tissue collected from PLEKHH2 gene heterozygous knockout rats three weeks after injection with MCT (40 mg/kg);

FIG. 23 is a diagram illustrating the binding between PLEKHH2 and Hic-5, and between SMAD7 and Hic-5 in the lung tissue of rats after hypoxia stimulation detected by Co-IP according to some embodiments of the present disclosure, where WT indicates wild-type rats housed under normoxia for three weeks as control; WT+Hypo indicates lung tissue collected from wild-type rats after three weeks of chronic hypoxia (an oxygen concentration of 10% (v/v)); and P-KO+Hypo indicates lung tissue collected from PLEKHH2 gene heterozygous knockout rats after three weeks of chronic hypoxia (an oxygen concentration of 10% (v/v));

FIG. 24 is a diagram illustrating the binding between PLEKHH2 and Hic-5, and between SMAD7 and Hic-5 in the lung tissue of rats with MCT-induced PAH after in vivo upregulation of PLEKHH2 expression detected by Co-IP according to some embodiments of the present disclosure, where WT indicates wild-type rats injected with normal saline; WT+MCT indicates wild-type rats 4 weeks after injection of MCT (50 mg/kg); and Ad-P+MCT indicates adenovirus prevention group, in which PLEKHH2 gene adenovirus was administered intratracheally via spray on the day of MCT injection to upregulate PLEKHH2 gene expression, a second administration was given at 2 weeks, and the experiment was terminated at 4 weeks;

FIG. 25 is a diagram illustrating the binding between PLEKHH2 and Hic-5, and between SMAD7 and Hic-5 in the lung tissue of a hypoxia and Sugen-induced PAH rat model after in vivo upregulation of PLEKHH2 expression detected by Co-IP according to some embodiments of the present disclosure, where WT indicates wild-type rats injected with normal saline and housed under normoxia for 5 weeks; WT+SuHx indicates a disease model group, wild-type rats subcutaneously injected with Su-5416 (20 mg/kg), exposed to hypoxia (an oxygen concentration of 10% (v/v)) for 3 weeks followed by reoxygenation for 2 weeks to establish a PAH rat model; and Ad-P+SuHx indicates a prevention group, in which PLEKHH2 gene adenovirus was administered intratracheally via spray on the day of subcutaneous injection of Su-5416 to upregulate PLEKHH2 gene expression, a second administration was given at 3 weeks, and the experiment was terminated at 5 weeks;

FIG. 26 is a diagram illustrating the activation of SMAD2/3 in the lung tissue of MCT-induced PAH rats after in vivo upregulation of PLEKHH2 expression detected by Western blot according to some embodiments of the present disclosure, where WT indicates wild-type rats injected with normal saline; WT+MCT indicates wild-type rats 4 weeks after injection of MCT (50 mg/kg); Ad-P+MCT indicates an adenovirus prevention group, in which PLEKHH2 gene adenovirus was administered intratracheally via spray on the day of MCT injection to upregulate PLEKHH2 gene expression, a second administration was given at 2 weeks, and the experiment was terminated at 4 weeks;

FIG. 27 is a diagram illustrating results of protein-protein interaction (PPI) network analysis related to PLEKHH2 according to some embodiments of the present disclosure, where Panel A illustrates a STRING database, and Panel B illustrates a GeneCards database;

FIG. 28 is a schematic diagram illustrating structures of PLEKHH2, Hic-5, and SMAD7 proteins according to some embodiments of the present disclosure;

FIGS. 29A-29C are diagrams illustrating molecular docking prediction of competitive binding of PLEKHH2 and SMAD7 to a LIM3 domain of a Hic-5 protein according to some embodiments of the present disclosure, where FIG. 29A illustrates a schematic diagram of binding of a N-terminal domain of SMAD7 to the LIM3 domain of Hic-5, with hydrogen bond interactions on the left and hydrophobic interactions on the right; FIG. 29B illustrates a schematic diagram of binding of a MH2 domain of SMAD7 to the LIM3 domain of Hic-5, with hydrogen bond interactions on the left and hydrophobic interactions on the right; and FIG. 29C illustrates a schematic diagram of binding of a FERM domain of PLEKHH2 to the LIM3 domain of Hic-5, with hydrogen bond interactions on the left and hydrophobic interactions on the right;

FIG. 30 is a schematic diagram illustrating structures of PLEKHH2, SMAD7, and Hic-5 according to some embodiments of the present disclosure, where green indicates PLEKHH2 protein, pink indicates SMAD7 protein, and blue indicates Hic-5 protein; purple indicates a binding region of the FERM domain of PLEKHH2, orange indicates a binding region of the MH2 domain of SMAD7, and yellow indicates an overlapping region;

FIG. 31 is an immunofluorescence image illustrating staining results in HPAECs according to some embodiments of the present disclosure, where blue indicates the nucleus stained with DAPI, green indicates the PLEKHH2 protein, red indicates the Hic-5 protein, and the ZOOM image shows a merged image of three channels;

FIG. 32 is an immunofluorescence image illustrating staining results in HPAECs according to some embodiments of the present disclosure, wherein blue indicates the nucleus stained with DAPI, red indicates the Hic-5 protein, white indicates the SMAD7 protein, and the ZOOM image shows the merged image of the three channels;

FIG. 33 is a diagram illustrating the effect of changes in PLEKHH2 expression on binding of PLEKHH2-Hic-5 and SMAD7-Hic-5 according to some embodiments of the present disclosure. HPAECs were cultured under normoxia and transfected with Ad-N, sh-P, and Ad-P for 48 hours, respectively. Hic-5 antibody-coated magnetic beads were used to enrich PLEKHH2 and SMAD7 proteins bound to Hic-5. Changes in an amount of bound protein and enriched samples were detected by Western Blot. Input indicates all non-enriched proteins in HPAECs; IgG indicates negative control for non-specific binding; Ad-N indicates HPAECs transfected with empty adenovirus vector; sh-P indicates HPAECs transfected with shRNA-PLEKHH2; Ad-P indicates HPAECs transfected with adenovirus-PLEKHH2; and HPAECs indicates Human pulmonary artery endothelial cells;

FIG. 34 is a diagram illustrating Hic-5 protein expression levels in the lung tissue of rat PAH models detected by Western Blot detection according to some embodiments of the present disclosure. The lung tissue from the same site was collected from the WT rats and SuHx model rats to extract total protein for detection, with a loading amount of 40 μg/well, and n=4. Data are presented as mean±standard error, and * indicates that P is less than 0.05;

FIG. 35 is a diagram illustrating Hic-5 expression levels in Human pulmonary artery smooth muscle cells (HPASMCs) after transfection with different siRNA sequences according to some embodiments of the present disclosure. One negative control sequence and five different Hic-5 knockdown siRNA sequences were transfected, and the knockdown efficiency indicates a degree of reduction in Hic-5 protein expression levels;

FIG. 36 is a diagram illustrating the effect of Hic-5 knockdown on the proliferation of HPASMCs according to some embodiments of the present disclosure. HPASMCs were seeded in 60 mm cell culture dishes and transfected with H-siR-1′ to knock down Hic-5. Subsequently, transfected cells were seeded into 24-well plates, and DNA replication in HPASMCs was detected using an Edu kit. Blue indicates nuclei stained with DAPI, green indicates Edu incorporation into DNA, and n=3. Data are presented as mean±standard error, and indicates that P is less than 0.001;

FIG. 37 is a chart illustrating the effect of Hic-5 knockdown on apoptosis of HPASMCs according to some embodiments of the present disclosure. HPASMCs were seeded in 60 mm cell culture dishes and transfected with H-siR-1′ to knock down Hic-5. Subsequently, transfected cells were seeded into 96-well plates, and Caspase 3/7 activity in the supernatant of HPASMC culture medium was detected using a Caspase-Glo 3/7 assay kit. The detected fluorescence intensity is positively correlated with the Caspase 3/7 activity in the supernatant, indicating late apoptosis and representing an apoptosis rate, and n=8. Data are presented as mean±standard error, and **** indicates that P is less than 0.0001;

FIG. 38 is a diagram illustrating the effect of Hic-5 knockdown on the migration ability of HPASMCs according to some embodiments of the present disclosure. HPASMCs were seeded in 60 mm cell culture dishes and transfected with H-siR-1′ to knock down Hic-5. Subsequently, transfected cells were seeded into 96-well plates. After scratching, continuous photographs were taken using an Incucyte imaging system to record HPASMCs migration within 3 hours. A wound healing rate was calculated based on a relative migration width, and n=4. Data are presented as mean±standard error, * indicates that P is less than 0.05, and ** indicates that P is less than 0.01;

FIG. 39 is a diagram illustrating that knockdown Hic-5 protein expression in pulmonary vessels via intratracheal spray of adenovirus si-Hic-5 effectively preventing MCT-induced PAH and SuHx-induced PAH according to some embodiments of the present disclosure, where panels above the dashed line show MCT stimulation, and panels below the dashed line show SuHx stimulation; panels A and C illustrate that RVSP and mPAPa are significantly elevated in the MCT-induced PAH model (MCT group), which are reduced by si-Hic-5 treatment; Panels E and G illustrate that RVSP and mPAP are also increased in a SuHx-induced PAH model (SuHx group), which were attenuated by si-Hic-5 treatment (SuHx+si-Hic-5 group); and Panels B, D, F, and H illustrate quantitative analysis of pulmonary artery pressure in different PAH models (n=8). Data are presented as mean±SD, * indicates that P is less than 0.05, ** indicates that Pis less than 0.01, *** indicates that P is less than 0.001, and **** indicates that P is less than 0.0001;

FIG. 40 is a diagram illustrating si-Hic-5 reducing PAH by regulating the BMP/TGF-β pathway according to some embodiments of the present disclosure. Western blot analysis was performed to detect the effect of Hic-5 knockdown on expression of key proteins in the BMPR2/TGF-β signaling pathway in the lung tissue of the MCT-induced PAH model and the SuHx-induced PAH model, main detected proteins include bone morphogenetic protein receptor type 2 (BMPR2), transforming growth factor-β (TGF-β), phosphorylated Small Mother Against Decapentaplegic 1/5/8 (P-SMAD1/5/8), Small Mother Against Decapentaplegic 1/5/8 (SMAD1/5/8), phosphorylated Small Mother Against Decapentaplegic 2/3 (P-SMAD2/3), Small Mother Against Decapentaplegic 2/3 (SMAD2/3), and ID1, and glyceraldehyde-3-phosphate dehydrogenase (GAPDH) was used as a reference protein. Data are presented as mean±SD, and n=4/8. * indicates that P is less than 0.05, ** indicates that P is less than 0.01, *** indicates that Pis less than 0.001, and ** indicates that P is less than 0.0001.

### DETAILED DESCRIPTION

Unless otherwise defined, all technical and scientific terms used herein have the same meaning as commonly understood by one of ordinary skill in the art to which the embodiments of the present disclosure pertain. To facilitate understanding of the embodiments of the present disclosure, the following terms involved in the embodiments of the present disclosure are explained herein:

As used herein, the term ‘including’ or ‘comprising’ refers to including any one or more of the recited elements or components, without excluding other elements or other components.

As used herein, the term ‘expression level’ or ‘level’ refers to an absolute amount or a relative amount of the biomarker PLEKHH2 described in the embodiments of the present disclosure, and the expression level of the biomarker PLEKHH2 described in the embodiments of the present disclosure can be determined by a plurality of techniques. For example, the absolute amount or the relative amount of the biomarker PLEKHH2 described in the embodiments of the present disclosure can be detected by using methods well known to a person skilled in the art (for example, real-time fluorescent quantitative PCR, mass spectrometry, or enzyme-linked immunosorbent assay).

As used herein, the term ‘primer’ refers to 7-50 nucleic acid sequences capable of forming a base pair complementary to a template strand and acting as a starting point for replicating the template strand. The primer is generally synthesized, but naturally occurring nucleic acids can also be used. The sequence of the primer does not necessarily need to be identical to the sequence of the template, as long as the sequence of the primer is sufficiently complementary to hybridize with the template. Additional features that do not alter the basic properties of the primer can be incorporated. For example, additional features that can be incorporated include methylation, capping, substitution of one or more nucleic acids by homologs, and internucleoside modification.

As used herein, the term ‘probe’ refers to a nucleic acid fragment from a few to hundreds of bases in length, such as RNA or DNA, which can establish a specific binding with a messenger RNA (mRNA) and can determine the presence of a specific mRNA due to a labeling action. The probe can be prepared in forms such as an oligonucleotide probe, a single-stranded DNA probe, a double-stranded DNA probe, and an RNA probe, etc.

As used herein, the term ‘antibody’ refers to a specific immunoglobulin against an antigen site. The antibody in the embodiments of the present disclosure refers to an antibody that specifically binds to the PLEKHH2 protein described in the embodiments of the present disclosure, and the antibody can be manufactured according to conventional methods in the art (for example, a rapid immunization method or a hybridoma technique). The forms of the antibody include a polyclonal antibody, a monoclonal antibody, antibody fragments (such as Fab fragments, Fab′ fragments, F(ab′) 2 fragments, and Fv fragments), a single-chain Fv (scFv) antibody, multi-specific antibodies (such as bispecific antibodies), a mono-specific antibody, a monovalent antibody, a chimeric antibody, a humanized antibody, a human antibody, a fusion protein including the antigen-binding site of the antibody, and any other modified immunoglobulin molecule including the antigen-binding site, as long as the antibody exhibits the desired biological binding activity.

As used herein, the term ‘peptide’ refers to a type of substance that has a high binding ability to a target substance (the biomarker protein described in the embodiments of the present disclosure) and is not denatured during heat treatment or chemical treatment. In addition, due to the small size of the peptide, it can be used as a fusion protein by attaching the peptide to other proteins. Specifically, because the peptide can be specifically attached to a macromolecular protein chain, the peptide can be used as a diagnostic kit and a drug delivery substance.

As used herein, the term ‘aptamer’ refers to a polynucleotide consisting of a specific type of single-stranded nucleic acid (DNA, RNA, or a modified nucleic acid), and the single-stranded nucleic acid has a stable tertiary structure and has a characteristic of binding to a target molecule (the biomarker protein described in the embodiments of the present disclosure) with high affinity and specificity. As described above, since the aptamer can specifically bind to an antigenic substance like an antibody, but is more stable and has a simpler structure than proteins, and consists of easily synthesized polynucleotides, the aptamer can be used instead of an antibody.

As used herein, the term ‘biomarker’ or ‘marker’ refers to an indicator of a phenotype of a patient (specifically referring to a PAH patient in the embodiments of the present disclosure), for example, an indicator of a pathological state or a potential responsiveness to a therapeutic agent, which can be detected in a biological sample of the patient, and the biomarker includes, but is not limited to, DNA, RNA, proteins, small molecule metabolites, carbohydrates, glycoplipid-based molecules, etc. In a specific embodiment of the present disclosure, the biomarker is PLEKHH2.

As used herein, the term ‘treatment’ generally involves treating a human or an animal (for example, as applied by a veterinarian), and certain desired therapeutic effects can be achieved, for example, inhibiting the progression of a condition (including reducing the rate of progression and halting the progression), ameliorating the condition, and curing the condition. The treatment also includes treatment as preventive measures (for example, prevention). Use in a patient who has not yet developed the condition but is at risk of developing the condition is also included in the term ‘treatment’.

As used herein, the term ‘diagnosis’ refers to the discovery, judgment, or cognition of a health state or condition of an individual based on one or more symptoms, data, or other information associated with the individual. The health state of the individual can be diagnosed as healthy/normal (i.e., absence of a disease or a disorder), or can be diagnosed as unhealthy/abnormal (i.e., presence of a disease or a disorder). The term diagnosis, early diagnosis, performing diagnosis, and variations thereof include early detection of a disease/condition associated with a specific disease or disorder (specifically referring to PAH in the embodiments of the present disclosure), characterization or classification of the disease, detection of progression, cure, or recurrence of the disease, and detection of a response to the disease after disposition or treatment of the individual. In the embodiments of the present disclosure, the diagnosis and/or auxiliary diagnosis of PAH includes distinguishing between an individual not suffering from PAH and an individual suffering from PAH.

As used herein, the term ‘pharmaceutical composition’ can have any one of the following preparations: a tablet, a pill, a powder, a granule, a capsule, a suspension, a solution, an emulsion, a syrup, a sterile aqueous solution, a non-aqueous solution, a lyophilized preparation, and a suppository. In addition, the pharmaceutical composition can be administered once or a plurality of times. In this case, the pharmaceutical composition can be administered in the form of a liquid preparation, a powder, an aerosol, a capsule, or a suppository.

The administration routes of the pharmaceutical composition include, but are not limited to, intraperitoneal, intravenous, intramuscular, subcutaneous, intradermal, oral, topical, intranasal, intrapulmonary, intrarectal, etc. When orally administered, it can be formulated with a coating that protects the active ingredient in the pharmaceutical composition from degradation in the stomach. In addition, the active ingredient can be administered by any device capable of transferring the pharmaceutical composition to a target tissue. The pharmaceutical composition provided in the embodiments of the present disclosure can be made into various dosage forms according to actual needs, and a beneficial dosage for a patient can be determined and administered by a clinician based on factors such as the type, the age, the weight, and the general disease condition of the subject, and the administration manner. The administration manner can be, for example, injection or any other suitable administration manner known to a person skilled in the art.

As used herein, the term ‘effective amount’ refers to an amount having a therapeutic effect or an amount required to produce a therapeutic effect in a subject. For example, a therapeutically or pharmaceutically effective amount refers to the amount of a drug required to produce a desired therapeutic effect, and the therapeutic effect can be reflected by results of clinical trials, model animal studies, and/or in vitro studies. The pharmaceutically effective amount depends on a plurality of factors, including but not limited to characteristic factors (such as a height, a weight, a gender, an age, and medication history) of the subject, the severity of the suffered disease, etc.

As used herein, the term ‘subject’ refers to any animal, including a human and a non-human animal. The non-human animal includes all vertebrates (e.g., mammals, such as non-human primates (e.g., higher primates), sheep, dogs, rodents (such as mice or rats), guinea pigs, goats, pigs, cats, rabbits, cattle, and any livestock or pets and non-mammals, such as chickens, amphibians, or reptiles).

In the embodiments of the present disclosure, by adopting a strategy of ‘genetic pedigree discovery-sporadic patient validation’, PLEKHH2 is firstly identified to be a novel PAH hereditary related gene, cell experiments show that the PLEKHH2 gene has an important protective effect on pulmonary vascular endothelial cells, and animal experiments confirm that high expression of PLEKHH2 can effectively prevent and treat PAH.

The embodiments of the present disclosure further explore downstream molecules and signaling pathways of the PLEKHH2 gene. Through a series of experiments such as virtual molecular docking, Co-IP, etc., Hic-5 is identified as a key downstream protein for PLEKHH2 to exert a protective effect. Under normal physiological conditions, both PLEKHH2 and SMAD7 (an endogenous TGF-β pathway inhibitory protein) competitively bind to the Hic-5 protein. When expression of the PLEKHH2 protein is decreased, its binding to Hic-5 is decreased, and the enhanced binding of Hic-5 to SMAD7 reduces the inhibitory effect of SMAD7 on TGF-β, which indicates that PLEKHH2 regulates the BMPR2/TGF-β signaling pathway balance by binding to Hic-5 under physiological conditions and maintains endothelial cell homeostasis. When the PLEKHH2 gene mutates or the expression of the PLEKHH2 gene is decreased, the PLEKHH2 gene loses the binding ability to Hic-5, Hic-5 captures more SMAD7 in the cytoplasm, which leads to the BMPR2/TGF-β pathway losing balance, causing endothelial cell hyperproliferation, mesenchymal transition, and pulmonary vascular pathological remodeling, which in turn leads to PAH.

A first aspect of the present disclosure provides a method for prevention and/or treatment of PAH, and the method includes administering to a subject an effective amount of at least one of a first agent that inhibits binding of Hic-5 to SMAD7 or a second agent that inhibits Hic-5.

The prevention refers to measures taken before the occurrence of a disease to prevent the occurrence of the disease or significantly reduce the risk of incidence.

Hic-5 is also referred to as TGFB111, and the corresponding Gene ID of Hic-5 in the NCBI (https://www.ncbi.nlm.nih.gov/) is 7041. The corresponding Gene ID of SMAD7 in the NCBI is 4092.

The first agent refers to an agent that inhibits the binding of Hic-5 to SMAD7.

In some embodiments, the first agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting a binding site between Hic-5 and SMAD7. In some embodiments, the binding site between Hic-5 and SMAD7 is a LIM3 domain of Hic-5.

In some embodiments, the first agent competitively binds to the LIM3 domain of Hic-5 with SMAD7.

In some embodiments, the agent (also referred to as the first agent) capable of competitively binding to the LIM3 domain of Hic-5 with SMAD7 comprises at least one of PLEKHH2 or a PLEKHH2 activator.

In some embodiments, the at least one of PLEKHH2 or the PLEKHH2 activator comprises at least one of a PLEKHH2 gene, a PLEKHH2 mRNA, a PLEKHH2 complementary DNA (cDNA), a PLEKHH2 protein, a peptide fragment of the PLEKHH2 protein, an analog of the PLEKHH2 protein, an active fragment of any of the foregoing, a vector expressing PLEKHH2, nanoparticles carrying the PLEKHH2 gene, a viral vector carrying the PLEKHH2 gene, a polyethylene glycol (PEG) ylated protein encapsulating the PLEKHH2 gene or the PLEKHH2 protein, protein microspheres encapsulating the PLEKHH2 gene or the PLEKHH2 protein, a liposome encapsulating the PLEKHH2 gene or the PLEKHH2 protein, or extracellular vesicles encapsulating the PLEKHH2 gene or the PLEKHH2 protein.

In some embodiments, the viral vector carrying the PLEKHH2 gene comprises but is not limited to an adenoviral vector, a lentiviral vector, a retroviral vector, a poxviral vector, a herpes simplex virus vector, or an adeno-associated viral vector.

In some embodiments, the at least one of PLEKHH2 or the PLEKHH2 activator prevents, treats, alleviates, and/or ameliorates the PAH by inhibiting the hyperphosphorylation of SMAD2/3.

Further description regarding PLEKHH2 and the PLEKHH2 activator can be found later in the present disclosure (e.g., the relevant description of the second aspect).

The second agent refers to an agent that inhibits Hic-5.

In some embodiments, the second agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting Hic-5.

In some embodiments, the second agent comprises a Hic-5 inhibitor. The Hic-5 inhibitor comprises but is not limited to an agent that inhibits or reduces the expression or transcription of a gene encoding a Hic-5 protein, or an agent that inhibits or reduces the expression or the activity of the Hic-5 protein. In some embodiments, the agent that inhibits or reduces the expression or the transcription of the gene encoding the Hic-5 protein comprises but is not limited to an agent that inhibits transcriptional activity of the Hic-5 gene, an agent that inhibits a transcription level of Hic-5 mRNA, an agent that promotes degradation of the Hic-5 mRNA, or an agent that specifically recognizes the Hic-5 gene with a guide nucleic acid and cleaves the Hic-5 gene to reduce the expression level of Hic-5. In some embodiments, the agent that inhibits or reduces the expression or the transcription of the gene encoding the Hic-5 protein comprises an siRNA targeting the Hic-5 gene, a short hairpin RNA (shRNA) targeting the Hic-5 gene, a double-stranded RNA (dsRNA) targeting the Hic-5 gene, a microRNA (miRNA) targeting the Hic-5 gene, an antisense nucleic acid targeting the Hic-5 gene, or a targeting vector targeting Hic-5. In some embodiments, the agent that inhibits or reduces the expression or the activity of the Hic-5 protein comprises an agent that inhibits translation of an Hic-5 mRNA, a specific antibody targeting Hic-5, or a small molecule compound that inhibits the activity of the Hic-5 protein.

In some embodiments, the activity of the Hic-5 protein may also be reduced by introducing a mutation into the Hic-5 protein. For example, a mutation that attenuates or abolishes the corresponding activity may be introduced into a functional domain of the Hic-5 protein. The mutation may be one or more amino acid insertions, deletions, or substitutions (e.g., more than 10, more than 20, or more than 30). The mutation that attenuates or abolishes the relevant biological activity may be introduced into the functional domain of the encoded Hic-5 protein by administering an agent that acts on the Hic-5 gene. The agent can change the sequence of the Hic-5 gene, leading to the corresponding mutation in the encoded Hic-5 protein, thereby exhibiting attenuated or abolished activity. For example, the wild-type Hic-5 gene may be replaced with a mutated Hic-5 gene through homologous recombination technology, thereby leading to the expression of a weakly active or inactive Hic-5 protein.

In some embodiments, the Hic-5 inhibitor is selected from a group comprising the siRNA targeting the Hic-5 gene, the shRNA targeting the Hic-5 gene, the agent that inhibits the transcriptional activity of the Hic-5 gene, the agent that inhibits the transcription level of the Hic-5 mRNA, the agent that promotes the degradation of the Hic-5 mRNA, the agent that inhibits the translation of the Hic-5 mRNA, the agent that specifically recognizes the Hic-5 gene with the guide nucleic acid and cleaves the Hic-5 gene to reduce the expression level of Hic-5, or a reagent for partial or complete knockout of the Hic-5 gene.

In some embodiments, the siRNA targeting the Hic-5 gene has a sequence shown in SEQ ID NOs: 9-10.

In some embodiments, the agent that inhibits the transcriptional activity of the Hic-5 gene may comprise a Histone Deacetylase (HDAC) inhibitor (e.g., trichostatin A).

In some embodiments, the agent that inhibits the transcription level of the Hic-5 mRNA may comprise a transcription inhibitor (e.g., actinomycin D).

In some embodiments, the agent that promotes the degradation of the Hic-5 mRNA may comprise an antisense oligonucleotide targeting Hic-5.

In some embodiments, the agent that inhibits the translation of the Hic-5 mRNA may comprise a translation inhibitor (e.g., puromycin or cycloheximide).

In some embodiments, the agent that specifically recognizes the Hic-5 gene with the guide nucleic acid and cleaves the Hic-5 gene to reduce the expression level of Hic-5 may comprise a CRISPR/CRISPR-associated protein 9 (Cas9) system, a CRISPR/CRISPR-associated protein 13 (Cas13) system, etc.

In some embodiments, the reagent for partial or complete knockout of the Hic-5 gene comprises the CRISPR/Cas9 system (e.g., Hic-5 CRISPR/Cas9 KO plasmid (h)).

In some embodiments, the Hic-5 inhibitor prevents, treats, alleviates, and/or ameliorates the PAH by restoring BMPR2-TGF-β pathway balance.

In some embodiments, the Hic-5 inhibitor or the second agent is capable of reducing the PAH, increasing BMPR2 pathway activity, reducing TGF-β pathway activity, or restoring the BMPR2-TGF-β pathway balance. In the embodiments of the present disclosure, it is experimentally verified for the first time that inhibiting the binding of Hic-5 to SMAD7 and/or inhibiting Hic-5 is effective for prevention and/or treatment of PAH.

The second aspect of the present disclosure provides a method for prevention and/or treatment of PAH, and the method comprises administering to a subject an effective amount of at least one of PLEKHH2 or the PLEKHH2 activator.

In some embodiments, the PLEKHH2 has a sequence known in the art or is a derivative molecule thereof. In some embodiments, the PLEKHH2 is a molecule as follows: (a) a PLEKHH2 molecule having a sequence shown in, for example, Gene ID: 130271 (human), Gene ID: 213556 (house mouse), Gene ID: 313866 (Norway rat), Gene ID: 713488 (rhesus monkey), or Gene ID: 100299044 (bovine); (b) a molecule that hybridizes to the sequence defined in (a) under stringent conditions; or (c) a molecule having at least 70% sequence identity (e.g., 75%, 80%, 85%, 90%, 95%, 98%, 99%, 99.5%, or more, or any value or range therebetween) to the sequence of the molecule shown in (a) or (b). The molecule includes a codon-optimized, species orthologous, or conservatively modified PLEKHH2 variant, as long as it retains PLEKHH2 functional activity.

In some embodiments, PLEKHH2 comprises the PLEKHH2 gene, the PLEKHH2 mRNA, the PLEKHH2 cDNA, the PLEKHH2 protein, the active fragment of any of the foregoing, or any combination thereof.

In some embodiments, the PLEKHH2 activator refers to a substance capable of increasing the PLEKHH2 level, enhancing the PLEKHH2 activity, and/or delaying the PLEKHH2 metabolism, comprises but is not limited to: a small molecule compound, the vector expressing PLEKHH2, the nanoparticles carrying the PLEKHH2 gene, the viral vector carrying the PLEKHH2 gene, the PEGylated protein encapsulating the PLEKHH2 gene or the PLEKHH2 protein, the protein microspheres encapsulating the PLEKHH2 gene or the PLEKHH2 protein, the liposome encapsulating the PLEKHH2 gene or the PLEKHH2 protein, the extracellular vesicles encapsulating the PLEKHH2 gene or the PLEKHH2 protein, or any combination thereof.

In some embodiments, the vector comprises but is not limited to: a lentiviral vector, a retroviral vector, a poxviral vector, a herpes simplex virus vector, an adenoviral vector, a adeno-associated viral vector, a DNA plasmid vector, a liposome coupled to a DNA plasmid, a molecular conjugate coupled to a DNA plasmid, and/or a polymer coupled to a DNA plasmid. Any vector capable of delivering the target gene PLEKHH2 as described in the embodiments of the present disclosure is within the scope of protection of the embodiments of the present disclosure. In some embodiments, the vector is the adenoviral vector.

In some embodiments, the PLEKHH2 activator comprises a substance that increases the PLEKHH2 level, a substance that enhances the PLEKHH2 activity, a substance that delays the PLEKHH2 metabolism, or any combination thereof.

In some embodiments, the PLEKHH2 activator comprises a naturally purified substance, a modified naturally purified substance, a semi-synthetic substance, and/or a chemically synthesized substance.

In some embodiments, the PLEKHH2 activator comprises the PLEKHH2 protein, the peptide fragment of the PLEKHH2 protein, the analog of the PLEKHH2 protein, the vector expressing PLEKHH2, the nanoparticles carrying the PLEKHH2 gene, the viral vector carrying the PLEKHH2 gene, the PEGylated protein encapsulating the PLEKHH2 gene or the PLEKHH2 protein, the protein microspheres encapsulating the PLEKHH2 gene or the PLEKHH2 protein, the liposome encapsulating the PLEKHH2 gene or the PLEKHH2 protein, the extracellular vesicles encapsulating the PLEKHH2 gene or the PLEKHH2 protein, or any combination thereof.

In some embodiments, the at least one of PLEKHH2 or the PLEKHH2 activator is capable of reducing the RVSP, alleviating the right ventricular hypertrophy, increasing the TAPSE, and/or alleviating the pulmonary arterial vascular remodeling.

In the embodiments of the present disclosure, the PLEKHH2 comprises the PLEKHH2 gene and the PLEKHH2 protein. The PLEKHH2 gene comprises a genomic DNA sequence located on a chromosome, including exons, introns, etc. The PLEKHH2 gene is transcribed and translated into the PLEKHH2 protein product in a subject. In some embodiments, the PLEKHH2 gene has a Gene ID of 130271, and the full name is pleckstrin homology, MyTH4 and FERM domain containing H2 [Homo sapiens (human)].

In some embodiments, the PLEKHH2 is from a mammal, including but not limited to: humans, non-human primates (e.g., orangutans, apes, and monkeys), rodents (e.g., rats, mice, and guinea pigs), pets (e.g., cats and dogs), and livestock (e.g., horses, cattle, sheep, pigs, and rabbits). In some embodiments, the PLEKHH2 is from the human.

A third aspect of the present disclosure provides a pharmaceutical composition for prevention and/or treatment of PAH, and the pharmaceutical composition comprises at least one of the first agent, the second agent, the PLEKHH2, or the PLEKHH2 activator as described above.

In some embodiments, the pharmaceutical composition may further comprise other drugs for prevention and/or treatment of the PAH. The other drugs for prevention and/or treatment of the PAH refer to drugs for prevention and/or treatment of the PAH other than at least one of the first agent, the second agent, the PLEKHH2, or the PLEKHH2 activator.

In some embodiments, the other drugs for prevention and/or treatment of the PAH comprise at least one of a calcium channel blocker, a prostacyclin analog, an endothelin receptor antagonist, a phosphodiesterase-5 inhibitor, or a guanylate cyclase stimulator. Any drug capable of being used for prevention and/or treatment of the PAH falls within the protection scope of the embodiments of the present disclosure.

In some embodiments, the calcium channel blocker comprises but is not limited to: diltiazem, verapamil, nifedipine, amlodipine, nitrendipine, felodipine, or lercanidipine. The prostacyclin analog comprises but is not limited to: rioprostil, abaprostil, dinoprostone, misoprostol, enprostil, trimoprostil, or roxaprostil. The endothelin receptor antagonist comprises but is not limited to: bosentan, ambrisentan, or macitentan. The phosphodiesterase-5 inhibitor comprises but is not limited to: tadalafil, vardenafil, or sildenafil. The guanylate cyclase stimulator comprises but is not limited to: riociguat, beraprost, vericiguat, or linaclotide.

In some embodiments, the pharmaceutical composition may further comprise at least one of a pharmaceutically acceptable carrier or an excipient. Specific examples of the pharmaceutically acceptable carrier and/or the excipient described in the embodiments of the present disclosure include but are not limited to: sugars (such as lactose, glucose, and sucrose), starches (such as corn starch and potato starch), cellulose and its derivatives (such as sodium carboxymethyl cellulose, ethyl cellulose, and methyl cellulose), tragacanth powder, malt, gelatin; talc, solid lubricants (such as stearic acid and magnesium stearate), calcium sulfate, vegetable oils (such as peanut oil, cottonseed oil, sesame oil, olive oil, corn oil, and cocoa butter), polyols (such as propylene glycol, glycerol, sorbitol, mannitol, and polyethylene glycol (PEG)), alginic acid, emulsifiers (such as wetting agents (such as sodium lauryl sulfate)), coloring agents, flavoring agents, tableting agents, stabilizers; antioxidants; preservatives, pyrogen-free water, isotonic saline solutions, phosphate buffers, or the like.

In some embodiments, the suitable pharmaceutically acceptable carrier and/or excipient are described in detail in Remington's Pharmaceutical Sciences (19th ed., 1995), and these substances are used as needed to help the stability of a formulation, or to help improve the activity or its bioavailability, or to produce an acceptable taste or odor in oral administration. The preparation that can be used in the pharmaceutical composition may be in the form of an original compound, or optionally in the form of a pharmaceutically acceptable salt. The pharmaceutical composition thus formulated may be administered as needed in any suitable manner known to the person skilled in the art. When using the pharmaceutical composition, the safe and effective amount of the pharmaceutical composition described in the embodiments of the present disclosure is administered to the subject.

A fourth aspect of the present disclosure provides the pharmaceutical preparation for prevention and/or treatment of PAH, and the pharmaceutical preparation comprises the pharmaceutical composition as described above.

In some embodiments, a dosage form of the pharmaceutical preparation comprises at least one of an injection, a lyophilized powder, a solution, a tablet, a capsule, a granule, an ointment, a cream, a gel, a suspension, an oral solution, a controlled-release formulation, a nanocrystal formulation, a microemulsion, or a solid dispersion. It should be noted that the specific dosage form of the pharmaceutical preparation is not particularly limited in the embodiments of the present disclosure, and any pharmaceutical dosage form known to the person skilled in the art is within the protection scope of the embodiments of the present disclosure.

In some embodiments, a suitable dosage of the pharmaceutical composition or the pharmaceutical preparation described in the embodiments of the present disclosure may be subjected to various prescriptions according to factors such as a formulation manner, the administration manner, a patient's age, a patient's weight, a patient's gender, a patient's disease state, a patient's diet, an administration time, an administration route, an excretion rate, and a reaction sensitivity. A skilled physician can usually easily determine the prescription and the effective dosage for the desired prevention and/or treatment.

In some embodiments, the administration manner is not particularly limited and includes but is not limited to local, transdermal, parenteral, intravenous, intramuscular, intraperitoneal, oral, intranasal dripping, intracavitary or intravesical dripping, intraocular, intra-arterial, intralesional, or administration to a mucous membrane such as those of the nose, throat, or bronchi.

A fifth aspect of the present disclosure provides a method for screening a candidate drug for prevention and/or treatment of PAH. The method comprises screening an agent that binds to a LIM3 domain of Hic-5 as the candidate drug; or simulating a binding site between Hic-5 and PLEKHH2, and screening an agent that binds to the binding site between Hic-5 and PLEKHH2 as the candidate drug; or simulating a binding site between Hic-5 and SMAD7, and screening an agent that binds to the binding site between Hic-5 and SMAD7 as the candidate drug; or treating a system expressing or containing PLEKHH2 gene with a test substance; detecting an expression level of the PLEKHH2 gene in the system; and selecting a test substance that increases the expression level of the PLEKHH2 gene as the candidate drug.

The method for screening a candidate drug for prevention and/or treatment of PAH based on the LIM3 domain of Hic-5 comprises the following steps: screening the agent that binds to the LIM3 domain of Hic-5 as the candidate drug.

The method for screening a candidate drug for prevention and/or treatment of PAH based on PLEKHH2 and/or Hic-5 comprises the following steps: simulating the binding site between Hic-5 and PLEKHH2 and designing and/or screening an agent that binds to the binding site between Hic-5 and PLEKHH2 as the candidate drug.

In some embodiments, the candidate drug competitively binds to Hic-5 with PLEKHH2. In some embodiments, the affinity of the candidate drug to Hic-5 is not lower than the affinity of PLEKHH2 to Hic-5, or the binding energy of the candidate drug to Hic-5 is not higher than the binding energy of PLEKHH2 to Hic-5. In some embodiments, the affinity of the candidate drug to Hic-5 is higher than the affinity of SMAD7 to Hic-5, or the binding energy of the candidate drug to Hic-5 is lower than the binding energy of PLEKHH2 to Hic-5.

The method for screening a candidate drug for prevention and/or treatment of PAH based on Hic-5 and/or SMAD7 comprises the following steps: simulating the binding site between Hic-5 and SMAD7 and designing and/or screening an agent that binds to the binding site between Hic-5 and SMAD7 as the candidate drug.

In some embodiments, the candidate drug competitively binds to Hic-5 with SMAD7. In some embodiments, the affinity of the candidate drug to Hic-5 is not lower than the affinity of SMAD7 to Hic-5, or the binding energy of the candidate drug to Hic-5 is not higher than the binding energy of SMAD7 to Hic-5. In some embodiments, the affinity of the candidate drug to Hic-5 is higher than the affinity of SMAD7 to Hic-5, or the binding energy of the candidate drug to Hic-5 is lower than the binding energy of SMAD7 to Hic-5.

The method for screening a candidate drug for prevention and/or treatment of PAH based on PLEKHH2 comprises the following steps: treating a system expressing or containing PLEKHH2 gene with a test substance; detecting an expression level of the PLEKHH2 gene in the system; and selecting a test substance that increases the expression level of the PLEKHH2 gene as the candidate drug.

In some embodiments, the method for screening a candidate drug for prevention and/or treatment of PAH based on the LIM3 domain of Hic-5 comprises: obtaining the LIM3 domain of Hic-5; screening structurally similar small molecule compounds in a molecular database based on a spatial structure of the LIM3 domain of Hic-5; performing molecular docking on screened small molecule compounds and the LIM3 domain of Hic-5 to determine the affinity or binding energy for targeting the LIM3 domain of Hic-5 to obtain scores; and sorting based on the scores to obtain the candidate drug. In some embodiments, the LIM3 domain of Hic-5 may be obtained by querying the Protein Data Bank (PDB).

In some embodiments, the method for screening a candidate drug for prevention and/or treatment of PAH based on PLEKHH2 and/or Hic-5 comprises: obtaining the binding site between PLEKHH2 protein and Hic-5 protein; screening structurally similar small molecule compounds in a molecular database based on the binding site between the PLEKHH2 protein and the Hic-5 protein; performing molecular docking on the screened small molecule compounds and the LIM3 domain of Hic-5 to determine the affinity or binding energy for targeting the LIM3 domain of Hic-5 to obtain scores; and sorting based on the scores to obtain the candidate drug.

In some embodiments, the method for screening a candidate drug for prevention and/or treatment of PAH based on Hic-5 and/or SMAD7 comprises: obtaining the binding site between Hic-5 protein and SMAD7 protein; screening structurally similar small molecule compounds in a molecular database based on the binding site between the Hic-5 protein and the SMAD7 protein; performing molecular docking on the screened small molecule compounds with the LIM3 domain of Hic-5 to determine the affinity or binding energy for targeting the LIM3 domain of Hic-5 to obtain scores; and sorting based on the scores to obtain the candidate drug.

In some embodiments, the method for screening a candidate drug for prevention and/or treatment of PAH based on PLEKHH2 comprises: treating a system expressing or containing PLEKHH2 gene with a test substance; detecting an expression level of the PLEKHH2 gene in the system; and selecting a test substance that increases the expression level of the PLEKHH2 gene as the candidate drug; wherein the system includes a cell system, a subcellular system, a solution system, a tissue system, an organ system, and/or an animal system.

A sixth aspect of the present disclosure provides a method for treating PAH. The method comprises the following steps: detecting an expression level of PLEKHH2 in a sample from a subject; and comparing the sample from the subject with a control sample, and administering an effective amount of at least one of PLEKHH2 or a PLEKHH2 activator to the subject in response to determining that an expression level of PLEKHH2 in the sample from the subject is lower than that in the control sample; wherein PLEKHH2 or the PLEKHH2 activator is as described above.

In some embodiments, the sample refers to a composition obtained or derived from the subject, which includes, for example, cellular entities and/or other molecular entities characterized and/or identified based on physical, biochemical, chemical, and/or physiological characteristics. The sample may be obtained from blood of the subject and other fluid samples and tissue samples of biological origin, such as a biopsy tissue sample or tissue cultures or cells derived therefrom. The source of the tissue sample may be solid tissue (such as fresh, frozen, and/or preserved organ or tissue samples, biopsy tissue or aspirates), blood or any blood component, body fluids, cells from any time of individual gestation or development, or plasma. The sample further includes processed biological samples, such as those treated with reagents, stabilized, or enriched for certain components (such as proteins or polynucleotides), or embedded in a semi-solid or solid matrix for sectioning purposes.

In some embodiments, the sample comprises but is not limited to blood, tissues, blood-derived cells, serum, plasma, lymph, synovial fluid, cells, cell extracts from the subject, and/or any combination thereof.

In some embodiments, the sample is selected from the blood or tissues of the subject.

In some embodiments, the detection of the expression level of PLEKHH2 in the sample comprises detecting the expression level of PLEKHH2 mRNA in the sample or detecting the expression level of PLEKHH2 protein in the sample. Techniques for detecting the expression level of PLEKHH2 in the sample include but are not limited to sequencing techniques, nucleic acid hybridization techniques, nucleic acid amplification techniques, or protein immunoassay techniques.

The control sample refers to a reference sample used for comparison with the sample from the subject, which is used for establishing a normal reference range for the PLEKHH2 expression level. The control sample may include a sample from a healthy individual or a non-diseased tissue sample from the subject. The healthy individual refers to an individual without the PAH, and without other known diseases or conditions that affect the PLEKHH2 expression.

A seventh aspect of the present disclosure provides a product for diagnosis and/or auxiliary diagnosis of PAH, and the product comprises a reagent for detecting an expression level of PLEKHH2 in a sample.

In some embodiments, the reagent comprises a reagent for detecting an expression level of PLEKHH2 mRNA in the sample, and a reagent for detecting an expression level of PLEKHH2 protein in the sample.

In some embodiments, the reagent for detecting the expression level of PLEKHH2 mRNA in the sample comprises a primer that specifically amplifies PLEKHH2 and/or a probe that specifically recognizes PLEKHH2.

In some embodiments, the reagent for detecting the expression level of PLEKHH2 protein in the sample comprises a binding agent that specifically binds to a protein encoded by PLEKHH2. The binding agent comprises but is not limited to an antibody that specifically binds to the PLEKHH2 protein, an antibody functional fragment, an agglutinant, a receptor, a conjugated antibody, an aptamer, and/or a compound.

In some embodiments, the product for diagnosis and/or auxiliary diagnosis of the PAH comprises a kit, a chip, and/or a test strip. The kit comprises but is not limited to a Reverse Transcription-Polymerase Chain Reaction (RT-PCR) kit, a DNA chip kit, an Enzyme-Linked Immunosorbent Assay (ELISA) kit, a protein chip kit, a rapid detection kit, or a Multiple Reaction Monitoring (MRM) kit.

In some embodiments, the kit is an RT-PCR kit, and the RT-PCR kit comprises components necessary for reverse transcription polymerase chain reaction. For example, the RT-PCR kit comprises a pair of primers specifically targeting a gene encoding a marker protein. Each primer is a nucleotide having a nucleic acid sequence specifically targeting the gene, a length of which may be about 7 to 50 base pairs (bp), more particularly about 10-39 bp. In some embodiments, the kit may comprise a primer having a nucleic acid sequence specifically targeting a control gene. In some embodiments, the RT-PCR kit may further comprise a test tube or a suitable vessel, a reaction buffer (with different pH values and magnesium concentrations), deoxyribonucleotides (dNTPs), enzymes (e.g., Taq polymerase and reverse transcriptase), a deoxyribonuclease inhibitor, a ribonuclease inhibitor, Diethylpyrocarbonate (DEPC)-treated water, or sterile water.

In some embodiments, the kit is a DNA chip kit, and the DNA chip kit comprises components necessary for operating a DNA chip. For example, the DNA chip kit may comprise a substrate bound to oligonucleotides corresponding to a gene, a cDNA, or a fragment thereof, and reagents, agents, and enzymes for constructing a fluorescently labeled probe. Furthermore, the substrate may comprise oligonucleotides corresponding to a control gene, a cDNA, or a fragment thereof.

In some embodiments, the kit is an ELISA kit, and the ELISA kit may comprise components necessary for performing an ELISA. For example, the ELISA kit may comprise an antibody specifically targeting a protein (the biomarker PLEKHH2 protein as described in embodiments of the present disclosure). The antibody has high selectivity and affinity for the biomarker protein, no cross-reactivity with other proteins, and may be a monoclonal antibody, a polyclonal antibody, or a recombinant antibody. Furthermore, the ELISA kit may comprise an antibody specifically targeting a control protein. In some embodiments, the ELISA kit may comprise a reagent capable of detecting a bound antibody (e.g., a labeled second antibody, a chromophore, an enzyme (e.g., an enzyme conjugated to the antibody) and a substrate thereof) or a substance capable of binding to the antibody.

An eighth aspect of the present disclosure provides a system and/or an apparatus for diagnosis and/or auxiliary diagnosis of PAH. The system and/or apparatus comprises a processor, an input module, and an output module. The processor is configured to perform logical operations on input information using a bioinformatics approach. The input module is configured to input an expression level of PLEKHH2 in a sample from a subject and includes a computer-readable medium containing instructions. The instructions, when executed by the processor, execute an algorithm on the input expression level of PLEKHH2. The output module is configured to output whether the subject suffers from PAH or has a risk of suffering from PAH. In some embodiments, the processor may comprise a central processing unit, a graphics processing unit, a Field-Programmable Gate Array (FPGA), or the like. In some embodiments, the input module may comprise a qPCR instrument, a microplate reader, a keyboard, a barcode scanner, or the like. In some embodiments, the output module may comprise a display, a printer, a speaker, or the like.

In some embodiments, the subject refers to any animal, including humans and non-human animals. The term ‘non-human animal’ includes all vertebrates (e.g., mammals, such as non-human primates (particularly higher primates), sheep, dogs, rodents (such as mice or rats), guinea pigs, goats, pigs, cats, rabbits, cattle, and any livestock or pets) and non-mammalian animals, such as chickens, amphibians, reptiles, or the like. In some embodiments, the subject is preferably a human.

Compared with the prior art, the embodiments of the present disclosure have the following beneficial effects.

(1) The PLEKHH2 is identified to be a novel PAH-related gene for the first time by adopting a strategy of ‘genetic pedigree discovery-sporadic patient validation’ in embodiments of the present disclosure. Results from gene functional studies indicate that PLEKHH2 is specifically expressed in PAECs and has potential functions of regulating BMPR2/TGF-β pathway activity and protecting the PAECs. Furthermore, the PLEKHH2 gene is significantly downregulated under PAH pathological conditions. Upregulating the expression of the PLEKHH2 gene with the adenovirus in the PAH animal model can effectively prevent and reverse PAH induced by MCT, which demonstrates the effectiveness of PLEKHH2 in the prevention, treatment, and/or diagnosis of PAH, and provides a new therapeutic target for the treatment of PAH, thus having significant clinical application value.

(2) The PLEKHH2 and SMAD7 competitively bind to the Hic-5 protein in the lung tissue in embodiments of the present disclosure. Under PAH pathological conditions, the PLEKHH2 protein mutates or its expression level decreases, and Hic-5 captures more SMAD7, which prevents SMAD7 from inhibiting SMAD2/3 activity, thereby enhancing SMAD2/3 signaling pathway activity, promoting hyperproliferation of endothelial cells, accelerating an endothelial-mesenchymal phenotypic transition, and ultimately leading to PAH. Upregulating PLEKHH2 expression can prevent Hic-5 from binding to SMAD7, cause more SMAD7 to be released into the cytoplasm, and inhibit SMAD2/3 phosphorylation, thereby treating PAH.

(3) A molecular mechanism by which PLEKHH2 protects against pulmonary vascular remodeling is clarified, and a plurality of novel approaches for treating PAH are proposed and validated in embodiments of the present disclosure.

1) Research results of embodiments of the present disclosure indicate that Hic-5 is highly expressed under PAH disease conditions, captures more SMAD7 protein, and prevents it from inhibiting the function of SMAD2/3. Therefore, degrading Hic-5 is a potential strategy for treating PAH.

2) Designing small molecule compounds that mimic a binding site between Hic-5 and PLEKHH2 to reduce binding between Hic-5 and SMAD7 represents a promising direction for novel drug development.

Hereinafter, in conjunction with specific examples, the present disclosure is further elaborated, which is provided only for explaining the present disclosure, and should not be construed as limiting the present disclosure. A person having ordinary skill in the art may understand that, without departing from the principles and spirit of the present disclosure, these embodiments may be subjected to various changes, modifications, substitutions, and variations, and the scope of the present disclosure is defined by the claims and equivalents thereof. In the following examples, experimental approaches for which specific conditions are not specified are generally conducted according to conventional conditions or conditions recommended by manufacturers.

### Example 1: Collection and Study of HPAH Pedigrees

### 1. Experimental Approaches

During the period from January 2021 to December 2022, a total of 10 HPAH pedigrees were collected from the Genetic Clinic of Fuwai Hospital, Chinese Academy of Medical Sciences. Whole exome sequencing (WES) was performed on the proband of each of the above-mentioned HPAH pedigrees to screen for mutations in 21 known PAH-causative genes (ACVRL1, BMPR2, ENG, GDF2, SMAD9, CAV1, ATP13A3, KCNK3, SOX17, EIF2AK4, TBX4, AQP1, SMAD4, SMAD1, KLF2, BMPRIB, KCNA5, ABCC8, KDR, TET2, and GGCX).

The approach for the WES was as follows. DNA was extracted from the collected whole blood of the subjects using a QIAamp® DNA kit (Qiagen, Germany), and WES was performed according to standards described in previous studies (see Reference 1: Wang X J, Lian T Y, Jiang X, et al. Germline BMP9 mutation causes idiopathic pulmonary arterial hypertension. The European respiratory journal 2019; 53; and Reference 2: Tan J S, Yan X X, Wu Y, et al. Rare variants in MTHFR predispose to occurrence and recurrence of pulmonary embolism. International journal of cardiology 2021; 331:236-42). A DNA library was constructed using a SureSelect Human All Exon V6 kit, and sequencing was performed using an Illumina NovaSeq 6000 system. The average coverage depth of the target region for each sample was more than 100×, and more than 90% of the target bases were sequenced more than 20 times.

### 2. Experimental Results

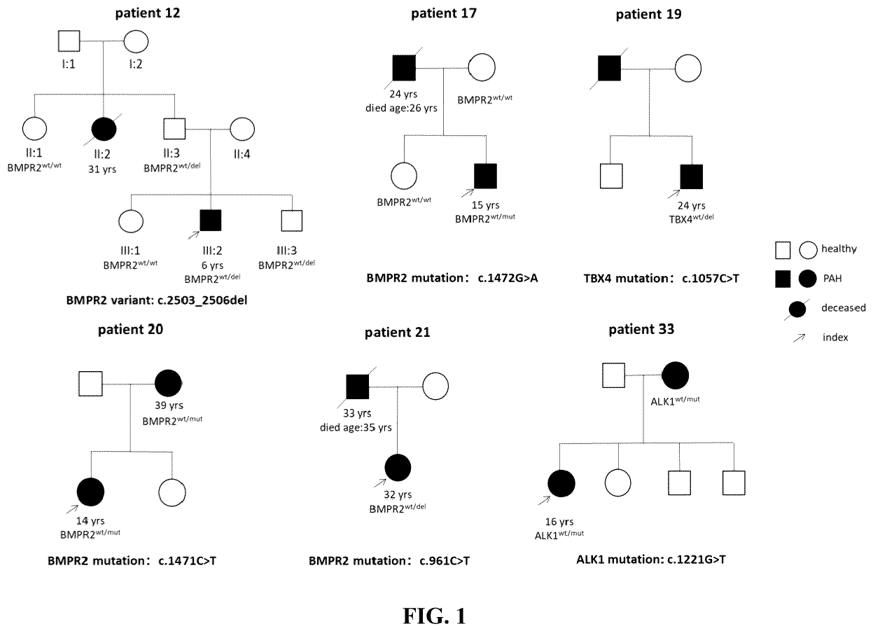

The results show that 6 pedigrees carry known PAH pathogenic mutations (see FIG. 1), including 4 BMPR2 mutations, 1 TBX4 mutation, and 1 ALK1 mutation (see Table 1), with a mutation detection rate of 60%.

TABLE 1
Pathogenic mutations identified in HPAH pedigrees

| | | | | | | Allele | | |
|---|---|---|---|---|---|---|---|---|
| | | | | | | frequency in | | American |
| | | | | | | 1000G_Chinese | | College |
| | | | | | | and | | of |
| | | | | | | Exome | | Medical |
| | | | | | | Aggregation | | Genetics |
| | | | | Amino | | Consortium | | and |
| | | | Nucleotide | acid | Mutation | East Asians | SIFT, | Genomics |
| Patient | Gene | Location | change | change | type | (ExAC_EAS) | PolyPhen2 | (ACMG) |
| 12 | BMPR2 | exon12 | c.2503_2506del | p.Thr835fs | Frameshift | Absent, | NA, NA | Pathogenic |
| | | | | | | Absent | | |
| 17 | BMPR2 | exon11 | c.1472G > A | p.Arg491Gln | Missense | Absent, | D, P | Pathogenic |
| | | | | | | Absent | | |
| 19 | TBX4 | exon10 | c.1057C > T | p.Arg353* | Nonsense | Absent, | NA, NA | Pathogenic |
| | | | | | | Absent | | |
| 20 | BMPR2 | exon11 | c.1471C > T | p.Arg491Trp | Missense | Absent, | D, P | Pathogenic |
| | | | | | | Absent | | |
| 21 | BMPR2 | exon7 | c.961C > T | p.Arg321* | Nonsense | Absent, | NA, NA | Pathogenic |
| | | | | | | Absent | | |
| 33 | ALK1 | exon7 | c.1221G > T | p.Glu407Asp | Missense | Absent, | D, D | Uncertain |
| | | | | | | Absent | | significance |

In the table, NA indicates that PolyPhen2 and SIFT are unable to analyze frameshift, nonsense, frameshift deletion, large fragment deletion types of variants, as well as variants in genes with unknown causes in electronic prediction programs, D indicates that variant is deleterious in SIFT and probably damaging in PolyPhen2, and P indicates that variant is probably damaging.

### Example 2: Association of PLEKHH2 Gene Mutation with Familial PAH

### 1. Experimental Approaches

For four pedigrees in which pathogenic mutations had not yet been identified, WES was performed on all core members of the 7th and 8th pedigrees to search for new potential causative genes.

The 7th pedigree included two PAH patients. The proband was a 42-year-old female. Right heart catheterization showed that the mPAP was 63 mmHg, the pulmonary vascular resistance (PVR) was 12.1 Wood Unit, and an acute pulmonary vasodilator test was negative. The proband's second son (21 years old) also had PAH. The mPAP was 87 mmHg, the PVR was 18.6 Wood Unit, and the acute pulmonary vasodilator test was negative. The proband's husband and the proband's eldest son were both healthy.

The 8th pedigree also had two PAH patients. The proband was a female who was diagnosed with PAH at 38 years old. The mPAP was 43 mmHg, the PVR was 5.3 Wood Unit, and the acute pulmonary vasodilator test was negative. The proband's daughter also had PAH. At 14 years old, the RVSP was assessed as 60 mmHg by echocardiography. The proband's husband was healthy.

In the present example, WES was performed on all four members from the 7th pedigree and all three members from the 8th pedigree. Rare deleterious variants were selected based on the following criteria:
(1) Co-segregation with the disease phenotype: the variant is shared by the two patients but absent in controls;
(2) The variant is located in a gene exonic region or an exon-intron splicing region;
(3) The variant type is missense, stop gain, stop loss, or small indel, and should affect amino acid encoding;
(4) The variant has a Minor Allele Frequency (MAF) of less than 0.1% in databases such as the 1000 Genomes Project and East Asian populations in Genome Aggregation Database (GnomAD);
(5) At least one of the three bioinformatics software tools, SIFT, PolyPhen-2, and Mutation taster, predicts the variant to be deleterious.

### 2. Experimental Results

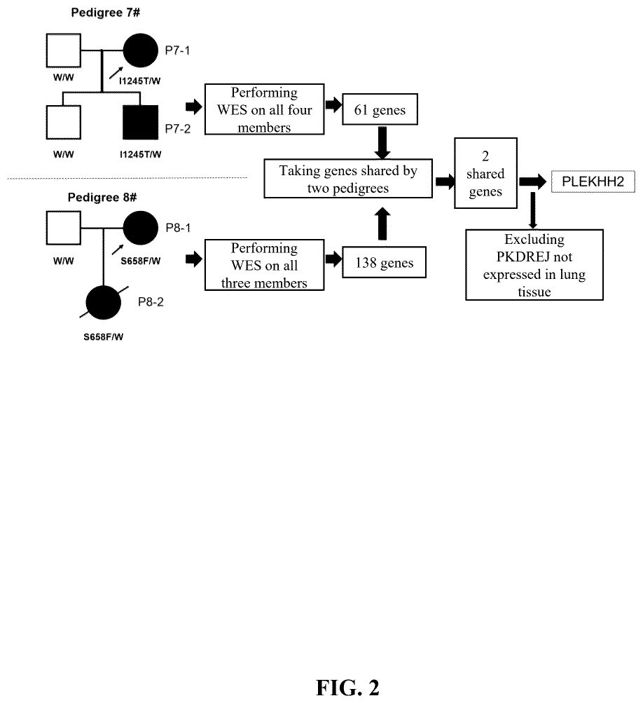

The results show that, after screening, a total of 61 genes is selected in the 7th pedigree, and a total of 138 genes is selected in the 8th pedigree. Intersection of rare variants from the two pedigrees is taken, only two genes appear repeatedly, namely PLEKHH2 and PKDREJ. Since PKDREJ is not expressed in the lung tissue, the gene is excluded, leaving only the PLEKHH2 gene (see FIG. 2).

### Example 3: Verification of Genetic Correlation Between PLEKHH2 Gene Mutation and PAH

To clarify a correlation between a PLEKHH2 gene mutation and pathogenesis of PAH, 176 IPAH patients were selected from Fuwai Hospital, Chinese Academy of Medical Sciences, and all of them underwent WES. Since all of the 176 patients were sporadic patients and had no family history as a reference, more stringent criteria were adopted when assessing pathogenicity of the PLEKHH2 gene genetic variants, specifically as follows:
(1) The variant is located in a gene exonic region or an exon-intron splicing region;
(2) The variant type is missense, stop gain, stop loss, or small indel, and should affect amino acid encoding;
(3) The variant frequency should be extremely low, requiring completely absence from the 1000 Genomes Project Chinese database and have a MAF of less than 1×10⁻⁵ in the East Asian population of the GnomAD;
(4) If the variant is a missense mutation, all three bioinformatics software tools, SIFT, PolyPhen-2, and Mutation taster, must predict the variant to be deleterious.

After adopting the above criteria, five newly identified patients were found to carry rare deleterious variants of the PLEKHH2 gene, with a mutation rate of 2.8% (5/176=2.8%). The clinical phenotypes of the five patients and the PLEKHH2 gene mutations carried are shown in Table 2. Specifically, the mutations included four missense mutations (c.1624C>T, c.1825G>A, c.2204A>G, c.3718A>T) and one truncating mutation (c.2389C>T). These mutations resulted in following protein sequence changes: p.P542S, p.A609T, p. Y735C, p.T1240S, and p.R797X (premature termination at amino acid position 797). The PLEKHH2 gene variants carried by the five patients were checked in the 1000 Genomes Project Chinese population and the GnomAD global population and found that the frequencies of all variants were less than 1×10⁻⁵ or completely unreported, which was consistent with the genetic characteristics of PAH as a rare disease. All four missense mutations were consistently judged as deleterious by the three bioinformatics analysis software tools. The variants of 21 known PAH pathogenic genes in the five patients were also analyzed, and it was found that they did not carry any known PAH pathogenic mutations.

Genetic burden of rare PLEKHH2 gene variants on PAH was then analyzed in the present example. Using the same criteria for screening rare variants in IPAH patients, the rare PLEKHH2 gene variants were retrieved in 76,156 controls from the GnomAD. 557 variants were identified, with a mutation rate of only 0.07%. Rare PLEKHH2 variants were significantly more prevalent in PAH patients (P=0.001, OR (Odds Ratio)=3.1). Thus, it is preliminarily confirmed that PLEKHH2 is a new genetic PAH-related gene and is closely related to the pathogenesis of PAH.

TABLE 2
Genotypes and clinical phenotypes of familial PAH patients
and IPAH patients carrying PLEKHH2 gene mutations

| | | | | | PVR | | Mutation | MAF in | |
|---|---|---|---|---|---|---|---|---|---|
| | Patient | | | mPAP | (Wood | CO | in | 1000G_C | MAF in |
| Group | ID | Age | Sex | (mmHg) | Unit) | (L/min) | PLEKHH2 | HB | GnomAD |
| HPAH | P7-1 | 42 | F | 63 | 12.1 | 4.1 | I1245T | NA | 10⁻⁶ |
| HPAH | P7-2 | 21 | M | 87 | 18.6 | 3.8 | I1245T | NA | 10⁻⁶ |
| HPAH | P8-1 | 38 | F | 43 | 5.3 | 6.4 | S658F | NA | 10⁻⁵ |
| HPAH | P8-2 | 14 | F | 60 | NA | NA | S658F | NA | 10⁻⁵ |
| IPAH | 61 | 18 | F | 32 | 6.0 | 5.1 | P542S | NA | NA |
| IPAH | 80 | 22 | F | 56 | 15.6 | 3.3 | A609T | NA | 10⁻⁶ |
| IPAH | 76 | 34 | F | 96 | 31.2 | 3.3 | Y735C | NA | NA |
| IPAH | 171 | 10 | M | 64 | 13.4* | 4.7 | R797X | NA | 10⁻⁶ |
| IPAH | 129 | 34 | F | 48 | 17.6 | 2.3 | T1240S | NA | NA |

In the table, HPAH indicates hereditary pulmonary arterial hypertension, IPAH indicates idiopathic pulmonary arterial hypertension, mPAP indicates mean pulmonary arterial pressure, PVR indicates pulmonary vascular resistance, CO indicates cardiac output, *indicates Patient No. 10 who is a child, and the value 13.4 here is the standardized Pulmonary Vascular Resistance index (PVRi), Sex: F is female, M is male, MAF in 1000G_CHB indicates a frequency of the variant in the 1000 Genomes Chinese population (387 individuals), and MAF in GnomAD indicates a frequency of the variant in the GnomAD (V3.1.1, approximately 76156 individuals).

### Example 4: Specific Expression of PLEKHH2 in PAECs

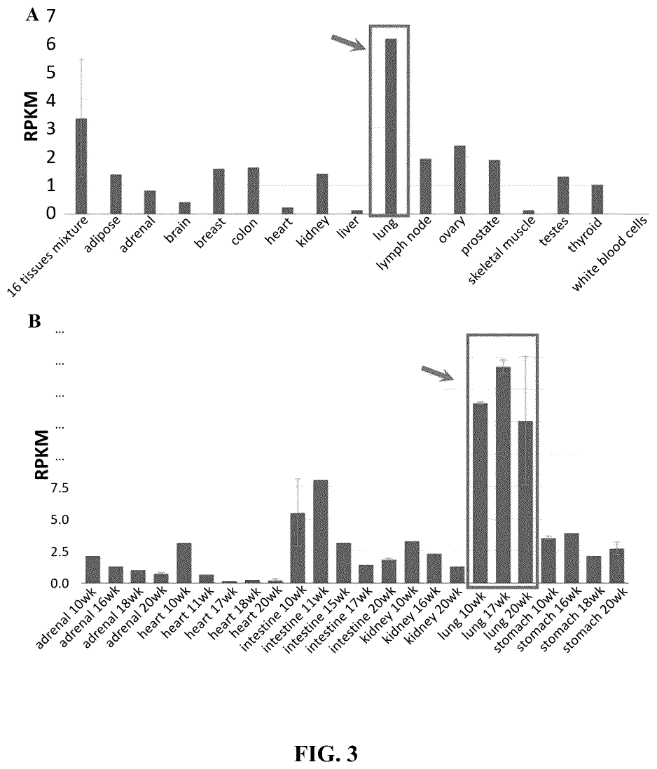

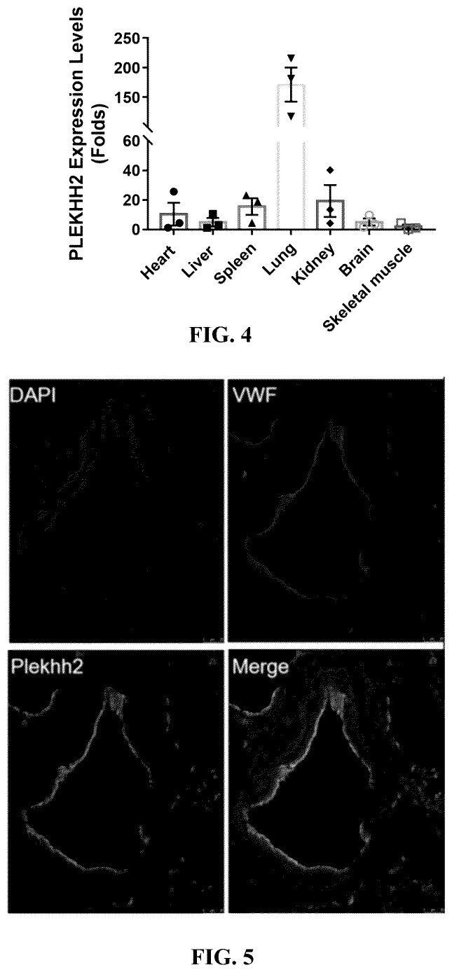

The NCBI database shows that during both a developmental stage and an adult stage, the PLEKHH2 gene is highly expressed in the lung tissue (see Panels A-B in FIG. 3). In this example, a plurality of organs (the heart, the lung, the liver, the spleen, the kidney, the brain, and the skeletal muscle) were obtained from two-month-old wild-type SD rats. Relative quantification was performed using GAPDH as an internal reference, and the expression of PLEKHH2 in a plurality of organ tissues of the rats was detected by qPCR. The results show that the PLEKHH2 gene is highly expressed in the lung tissue (see FIG. 4). The specific primer sequences used in the qPCR experiment are as follows:

PLEKHH2 gene:
(SEQ ID NO: 1)
The forward primer sequence is 5′-AGCGGACGACTCAAGA
CT-3′,
and
(SEQ ID NO: 2)
the reverse primer sequence is 5′-TGAACCGTTTGCTTGT
TA-3′.
GAPDH gene:
(SEQ ID NO: 3)
The forward primer sequence is 5′-CTCATGACCACAGTCC
ATGC-3′,
and
(SEQ ID NO: 4)
the reverse primer sequence is 5′-CACATTGGGGGTAGGA
ACAC-3′.

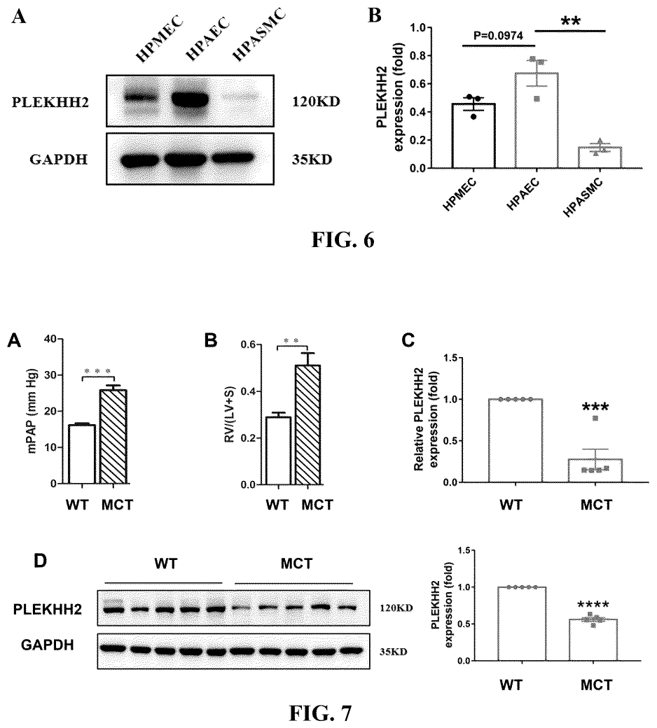

In this example, immunofluorescence was used to label the PLEKHH2 protein in human lung biopsy tissue. It was found that the PLEKHH2 protein was mainly localized in human pulmonary vascular endothelial cells, where blue indicates nuclear stained with DAPI, red indicates VWF (marking endothelial cells), and green indicates the PLEKHH2 protein (see FIG. 5). Furthermore, in this example, three types of human pulmonary vascular structural cells, including PASMCs, PMECs, and PAECs, were cultured in vitro. Western blot was performed to detect the expression level of the PLEKHH2 protein in the three types of cells. The results show that the PLEKHH2 protein is highly expressed in the two types of endothelial cells and is hardly expressed in the smooth muscle cells (see Panels A-B in FIG. 6). The characteristics of the high expression of PLEKHH2 in the lung tissue and its main localization in the pulmonary vascular endothelial cells further suggest that PLEKHH2 is closely related to pulmonary vascular pathophysiology.

### Example 5: Significant Reduction of PLEKHH2 Expression Under Pathological Conditions of PAH

### 1. Experimental Approaches

To determine whether PLEKHH2 is involved in the pathological process of PAH, the expression of the PLEKHH2 gene in two types of animal models of PAH was detected in this example.

First, a PAH rat model (also referred to as a MCT group) was established by subcutaneously injecting rats with 60 mg/kg MCT. After three weeks, the mPAP and the RV/(LV+S) of wild-type control rats (also referred to as a WT group) and the MCT group rats were detected. Additionally, the expression level of PLEKHH2 mRNA and the expression level of the PLEKHH2 protein in the lung tissue of the wild-type control rats and the MCT group rats were detected by qPCR and Western blot, respectively.

Next, a PAH rat model (also referred to as a SuHx group) was established by subcutaneously injecting rats with Su-5416 (20 mg/kg), followed by three weeks of hypoxia and two weeks of reoxygenation. After five weeks, the mPAP and the RV/(LV+S) of the wild-type control rats (the WT group) and the SuHx group rats were detected. Additionally, the expression level of PLEKHH2 mRNA and the expression level of the PLEKHH2 protein in the lung tissue of the wild-type control rats and the SuHx group rats were detected by qPCR and Western blot, respectively.

Finally, the lung tissue from patients with congenital heart disease combined with severe PAH and control lung tissue from subjects without PAH were further collected in this example. The expression of the PLEKHH2 gene in endothelial cells of the lung tissue from PAH patients was detected by immunofluorescence.

### 2. Experimental Results

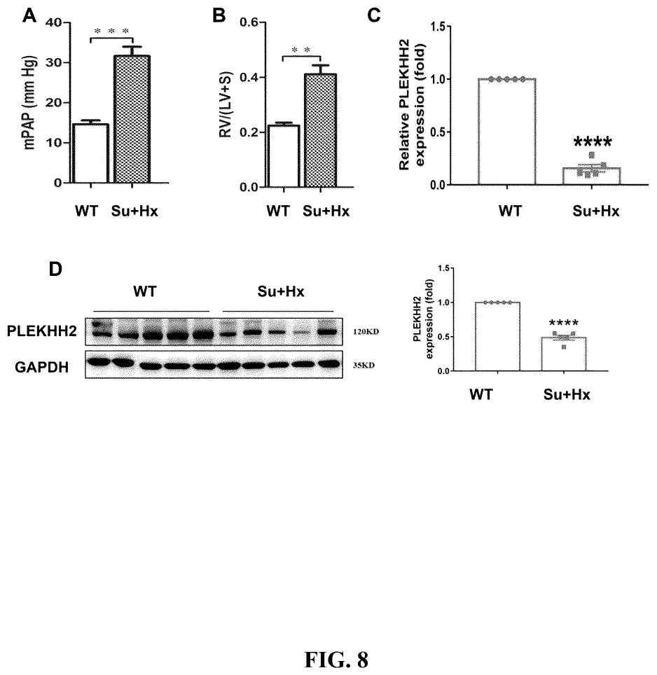

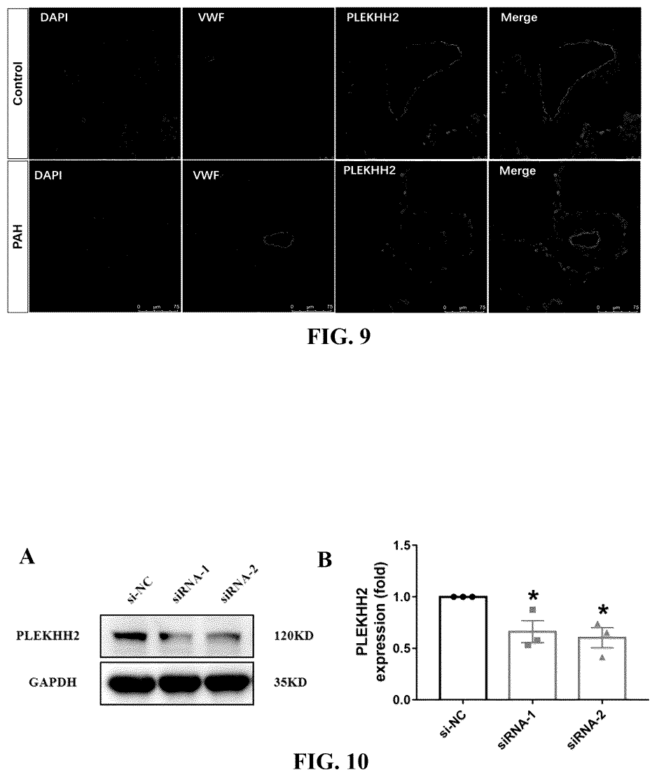

The results show that, in both the MCT-induced PAH rats and the hypoxia+Sugen-induced PAH rats, the expression of the PLEKHH2 gene is significantly reduced in the lung tissue of both types of the PAH rat models. Specifically, compared with the wild-type control rats, the mPAP and RV/(LV+S) of the MCT-induced PAH rats are significantly increased (see Panels A-B in FIG. 7). The expression of PLEKHH2 mRNA in the MCT group decreases by 62% (see Panel C in FIG. 7), and the protein expression decreases by 43% (see Panel D in FIG. 7), showing extremely significant differences. Furthermore, compared with the wild-type control rats, the mPAP and RV/(LV+S) of the hypoxia+Sugen-induced PAH rats are significantly increased (see Panels A-B in FIG. 8). The expression of PLEKHH2 mRNA in the hypoxia+Sugen group decreases by 76% (see Panel C in FIG. 8), and the protein expression decreases by 50% (see Panel D in FIG. 8), showing extremely significant differences. Furthermore, PLEKHH2 is also significantly reduced in the biopsy lung tissue of the PAH patients (see FIG. 9), suggesting that the gene is closely related to PAH pathological remodeling.

### Example 6 Endothelial-Mesenchymal Transition (EndoMT) in PAECs Induced by PLEKHH2 Gene Knockdown

### 1. Experimental Approaches

Since the expression of the PLEKHH2 gene is significantly reduced under pathological conditions of PAH, in this example, PAECs, which have the highest baseline expression of the PLEKHH2 protein, were selected as model cells. siRNA was used to knock down the PLEKHH2 protein to investigate the effect of PLEKHH2 on endothelial cell phenotype.

First, two siRNAs (i.e., siRNA-1 and siRNA-2) with different sequences targeting the PLEKHH2 gene were designed and synthesized in this example. Then, after transfecting the PAECs with the siRNAs, the expression level of the PLEKHH2 protein in the transfected PAECs was detected by Western blot. Next, the effect of the PLEKHH2 gene knockdown on the proliferation ability of the PAECs under the normoxic (an oxygen concentration of 20% (v/v)) condition (Normo) and the hypoxic (an oxygen concentration of 3% (v/v)) condition (Hypo) was detected by EDU cell proliferation assay in this example. Furthermore, the effect of PLEKHH2 knockdown on the expression of B-cell lymphoma 2 (Bcl-2) and Bcl-2-associated X protein (Bax) in the PAECs under normoxic conditions was detected by Western blot in this example. Finally, the effect of the PLEKHH2 gene knockdown on the tube formation ability of the PAECs under the normoxic (the oxygen concentration of 20% (v/v)) condition (Normo) and the hypoxic (the oxygen concentration of 3% (v/v)) condition (Hypo) was detected by Matrigel angiogenesis assay in this example.

The sequence information of siRNA-1 and siRNA-2 is as follows.

siRNA-1:
(SEQ ID NO: 5)
The sense strand sequence is 5′-GAGGAAAUGAGCAAGAUA
UTT-3′,
and
(SEQ ID NO: 6)
the antisense strand sequence is 5′-AUAUCUUGCUCAUU
UCCUCTT-3′.
siRNA-2:
(SEQ ID NO: 7)
The sense strand sequence is 5′-GGCUUCUGAAAGUGAUUA
UTT-3′,
and
(SEQ ID NO: 8)
the antisense strand sequence is 5′-AUAAUCACUUUCAG
AAGCCTT-3′

### 2. Experimental Results

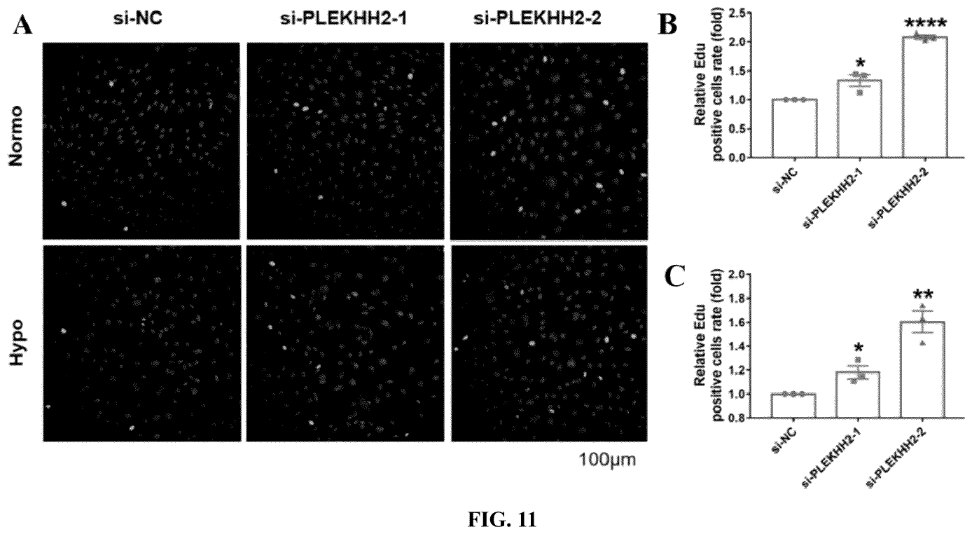

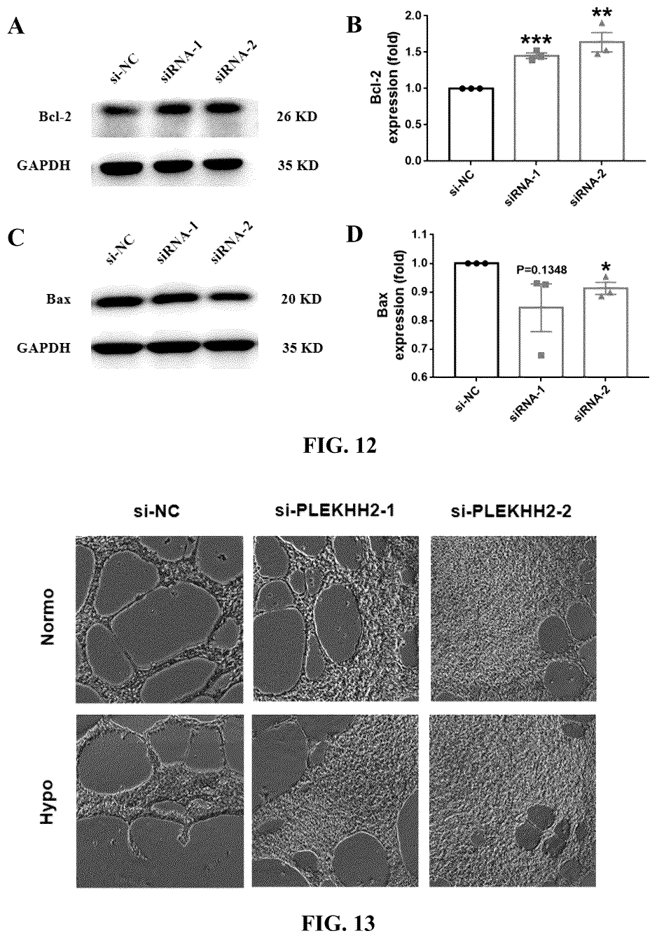

The results show that, after transfecting the PAECs with siRNA-PLEKHH2, the expression level of the PLEKHH2 gene is significantly reduced. The two interfering fragments, siRNA-1 and siRNA-2, decrease the expression of the PLEKHH2 protein by 40%-50% (see Panels A-B in FIG. 10). Under both the normoxic (the oxygen concentration of 20% (v/v)) condition or the hypoxic (the oxygen concentration of 3% (v/v)) condition, the proliferation ability of the PAECs is significantly increased after knocking down the PLEKHH2 gene (see Panels A-C in FIG. 11). The results of cell proliferation and apoptosis-related pathway proteins detected by Western blot show that a decrease in PLEKHH2 gene expression leads to a significant increase in an anti-apoptotic protein Bcl-2, while the expression of a pro-apoptotic protein Bax is reduced (see Panels A-D in FIG. 12). The results of the endothelial cell tube formation assay show that, under both the normoxic and hypoxic conditions, the tube formation ability of the siRNA-1 group with the PLEKHH2 gene knockdown is significantly decreased, and the siRNA-2 group could hardly form tubes (see FIG. 13). In summary, the PLEKHH2 gene knockdown can cause the PAECs to over-proliferate, resist apoptosis, and have difficulty forming tubes, leading to a significant EndoMT.

### Example 7: Significant Reduction of Endothelial Cell BMPR2 Pathway Activity after PLEKHH2 Gene Knockdown

### 1. Experimental Approaches

To verify the relationship between PLEKHH2 and the BMPR2/TGF-β pathway, in this example, Western blot was used to detect changes in the BMPR2/TGF-β pathway activity after PLEKHH2 knockdown in endothelial cells (PAECs), which included the detection of changes in the expression and phosphorylation of key proteins (BMPR2, P-SMAD1/5/8, SMAD1/5/8, P-SMAD2/3, SMAD2/3, and ID1) in the BMPR2/TGF-β pathway. In this example, siRNA-2, which has a better effect of PLEKHH2 knockdown in Example 6, was selected to investigate the molecular mechanism.

### 2. Experimental Results

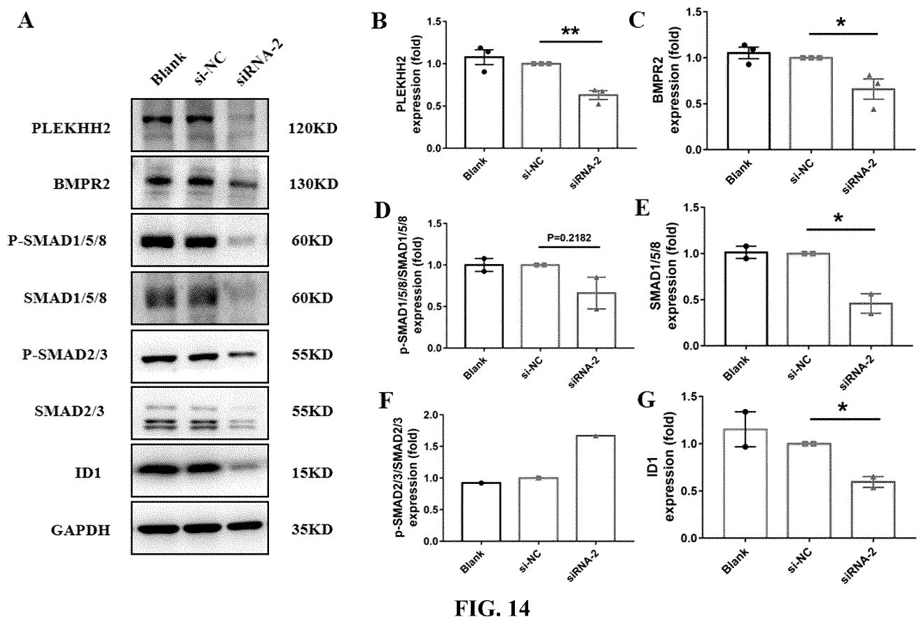

The results show that a decrease in PLEKHH2 expression leads to downregulation of BMPR2 expression in endothelial cells, reduced phosphorylation of SMAD1/5/8, and reduced ID1 expression. On the other hand, phosphorylation of SMAD2/3 in the TGF-β pathway is significantly increased (see Panels A-G in FIG. 14). Therefore, PLEKHH2 is likely a key gene regulating the BMPR2-TGF-β pathway balance in endothelial cells. PLEKHH2 knockdown leads to weakened BMPR2 signaling and enhanced TGF-β signaling, thereby leading to PAH vascular remodeling.

### Example 8: Effective Prevention of PAH by In Vivo Upregulation of PLEKHH2 Gene

### 1. Experimental Approaches

Both PLEKHH2 mutation or reduced expression can promote the occurrence and development of PAH, suggesting that PLEKHH2 should be a ‘protective gene’ for pulmonary vessels. In this example, an in vivo animal model was used to further investigate whether upregulation of PLEKHH2 could prevent PAH.

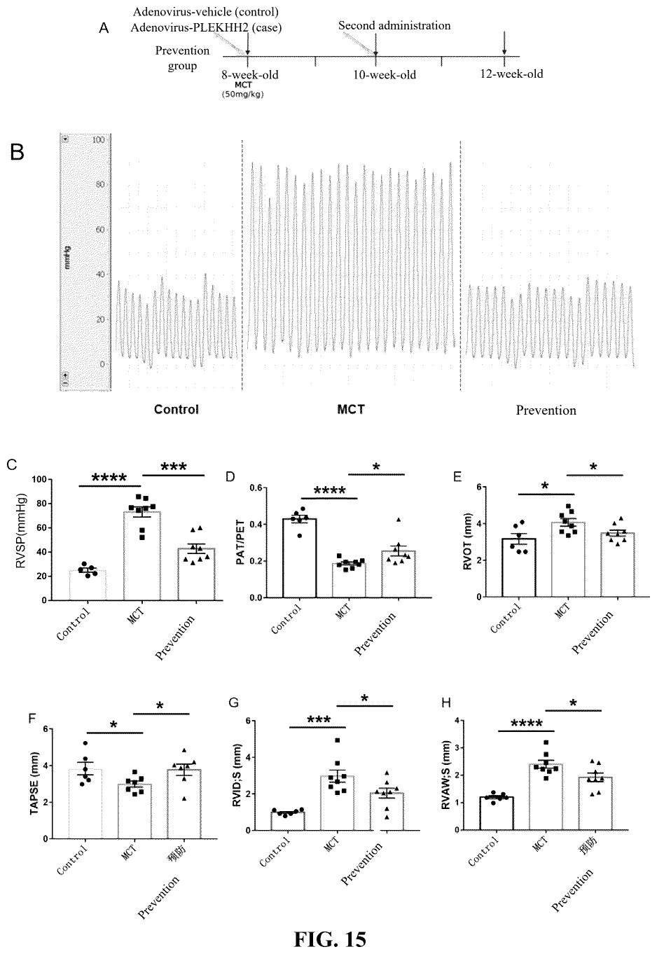

A PAH rat model was established by intraperitoneal injection of MCT (50 mg/kg) in 8-week-old rats. On the day of MCT injection, the PLEKHH2 gene adenovirus (adenovirus-PLEKHH2) was administered to the rats for the first time via intratracheal spray to upregulate PLEKHH2 gene expression. At 10 weeks of age, the PLEKHH2 gene adenovirus (adenovirus-PLEKHH2) was administered for the second time. The experiment was terminated at 12 weeks of age and the preventive effect of upregulating the expression of the PLEKHH2 gene on the MCT-induced PAH rats was evaluated. A schematic diagram of the specific experimental design is shown in Panel A in FIG. 15, where the control group is adenovirus-vehicle, and the case group is adenovirus-PLEKHH2. It is worth noting that the activity of the adenovirus can only be maintained in rats for two weeks. Therefore, in the four-week period of the present experiment, the adenovirus needs to be injected twice separately to observe the preventive effect.

### 2. Experimental Results

The results show that preventive administration of PLEKHH2 significantly reduces the right ventricular pressure of the MCT-induced PAH rats (see Panels B-C in FIG. 15), and the right ventricular wall thickness, the RVID, and the RVOT are all significantly reduced, and the TAPSE is significantly increased (see Panels D-H in FIG. 15), indicating that high expression of PLEKHH2 can effectively prevent the MCT-induced PAH.

### Example 9: Effective Treatment of PAH by In Vivo Upregulation of PLEKHH2 Gene

### 1. Experimental Approaches

After confirming that the high expression of PLEKHH2 has the function of preventing PAH, this example further investigated whether PLEKHH2 could treat PAH.

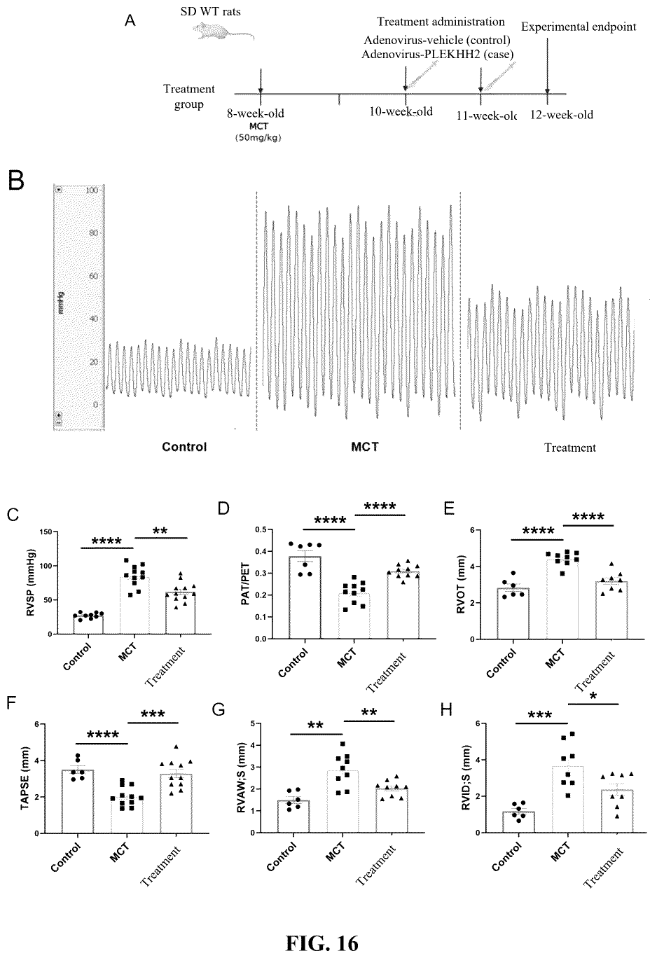

A PAH rat model was established by intraperitoneal injection of MCT (50 mg/kg) in 8-week-old rats. At the second week after the MCT injection, the pulmonary arterial pressure in the rats had already increased. The PLEKHH2 gene adenovirus (adenovirus-PLEKHH2) was administered to the rats by intratracheal spray. At the third week, the PLEKHH2 gene adenovirus was administered again. At the fourth week, the experiment was terminated and the therapeutic effect of upregulating the expression of the PLEKHH2 gene on the MCT-induced PAH rats was evaluated. A schematic diagram of the specific experimental design is shown in Panel A in FIG. 16, where the control group is adenovirus-vehicle, and the case group is adenovirus-PLEKHH2.

### 2. Experimental Results

The results show that therapeutic administration of PLEKHH2 significantly reduces the already existing pulmonary arterial pressure in the MCT-induced PAH rats (see Panels B-C in FIG. 16), and the right ventricular wall thickness, the RVID, and the RVOT are all significantly reduced, and the TAPSE was significantly increased (see FIGS. 16D-F), indicating that high expression of PLEKHH2 can effectively treat the MCT-induced PAH.

### Example 10: Decreased Expression of PLEKHH2 and SMAD7 and Increased Expression of Hic-5 in a PAH Rat Model

The roles of Hic-5 and SMAD7 in PAH are currently completely unknown. To clarify whether the two proteins participate in the pathological process of PAH, the present example constructed a classical MCT-induced PAH animal model using conventional manners, and detected the expression of PLEKHH2, Hic-5, and SMAD7 proteins.

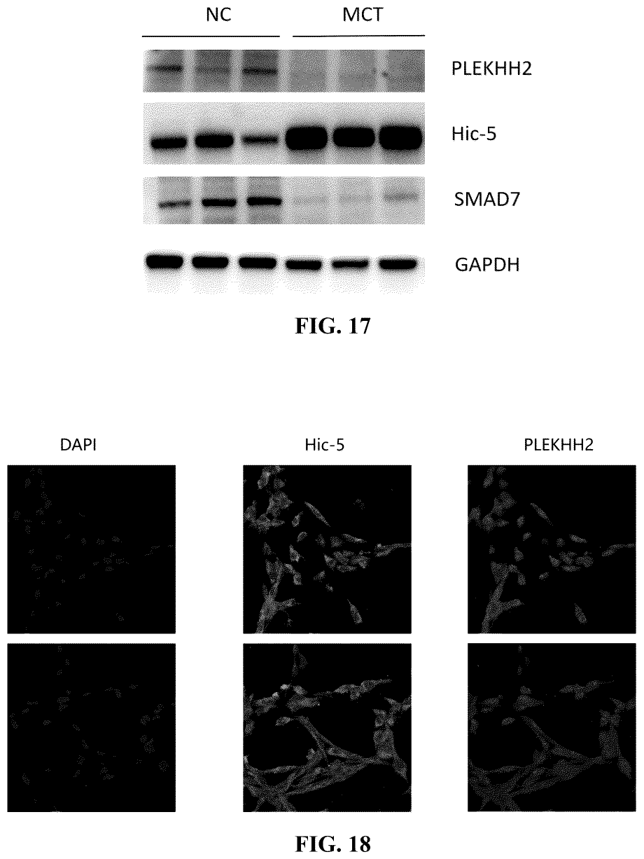

The PAH rat model (MCT group) was established by subcutaneously injecting 60 mg/kg MCT into rats, with subcutaneous injection of normal saline as a control group. After three weeks, the lung tissues were collected from the control group rats and the MCT group rats separately for Western blot analysis (FIG. 17). The results show that in the MCT model group, the PLEKHH2 protein is extremely significantly decreased (this result is completely consistent with the results in the foregoing examples), the Hic-5 expression increases, and the SMAD7 expression decreases, suggesting that all three proteins participate in PAH vascular remodeling.

### Example 11: Colocalization of PLEKHH2 and Hic-5 in Pulmonary Vascular Endothelial Cells

Previous literature reported that PLEKHH2 and Hic-5 colocalize in podocytes (i.e., visceral epithelial cells of the renal capsule), but it is completely unknown whether colocalization exists in pulmonary vascular endothelial cells.

To this end, the PAECs were cultured in vitro and the immunofluorescence staining experiments confirm that both Hic-5 and PLEKHH2 are highly expressed in the PAECs, their subcellular localization is mainly concentrated in the cytoplasm, and the localization of the two proteins shows a high degree of overlap (FIG. 18).

### Example 12: Bioinformatics Prediction of Competitive Binding of PLEKHH2 and SMAD7 to LIM3 Domain of Hic-5

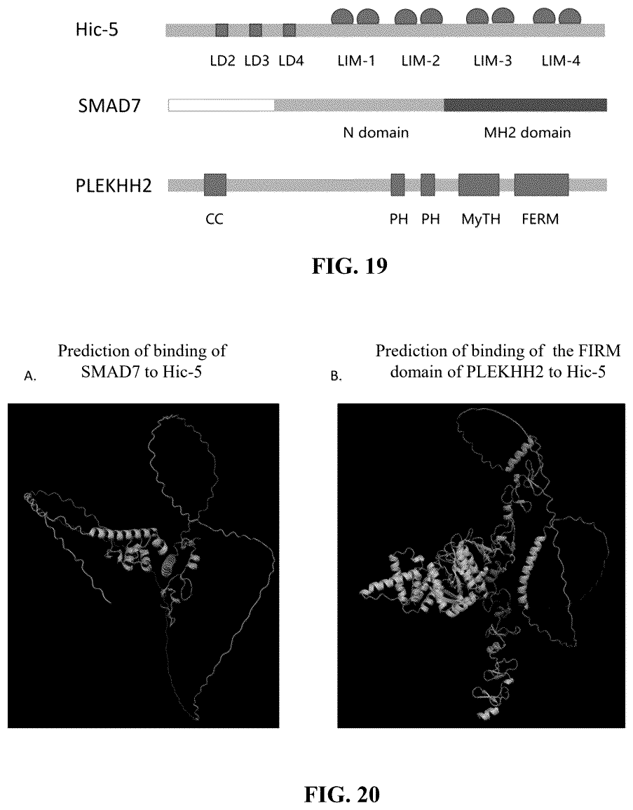

The Hic-5 protein has seven main functional domains: namely, three LD domains and four LIM domains. SMAD7 has two main domains: an N-terminal domain and an MH2 domain. PLEKHH2 has five domains: a CC domain, two PH domains, a MyTH domain, and a FIRM domain (FIG. 19).

It has been reported in the literature that the MH2 domain and N-terminal domain of the SMAD7 protein directly bind to the LIM3 domain (328-380aa) of the Hic-5 protein (Oncogene. 2008 Nov. 20; 27 (54): 6791-6805). The binding ability of the above-mentioned two protein domains was verified using the Alpha-Fold-3 software (Panel A in FIG. 20).

According to the literature, the FIRM domain of PLEKHH2 can bind to Hic-5 (Kidney International (2012) 82, 1071-1083), but it is not yet clear which domain of Hic-5 it specifically binds to.

To this end, the FIRM domain of PLEKHH2 and the full-length protein sequence of Hic-5 were inputted into the Alpha-Fold-3 software and it is found that the FIRM domain of PLEKHH2 also directly binds to the LIM3 domain of Hic-5. Furthermore, the protein sequence of Hic-5 binding to SMAD7 was marked in purple, and this location was precisely within a binding interface between the FIRM domain of PLEKHH2 and the LIM3 domain of Hic-5 (Panel B in FIG. 20), which suggests that both PLEKHH2 and SMAD7 are likely to bind to the LIM3 region of Hic-5, and a competitive binding relationship exists between the two.

### Example 13: Competitive Binding of PLEKHH2 and SMAD7 to Hic-5 at the Cellular Level

To clarify whether PLEKHH2 and SMAD7 competitively bind to Hic-5, the pulmonary vascular endothelial cells were cultured, the PLEKHH2 gene (sh-P) was knocked down using an shRNA, and the siRNA sequences generated by intracellular processing of sh-P are shown as SEQ ID NO: 5 and SEQ ID NO: 6. The PLEKHH2 gene (Ad-P) was overexpressed using an adenovirus, and then a Co-IP experiment was used to determine the changes in the binding Hic-5 to SMAD7 after PLEKHH2 expression was altered.

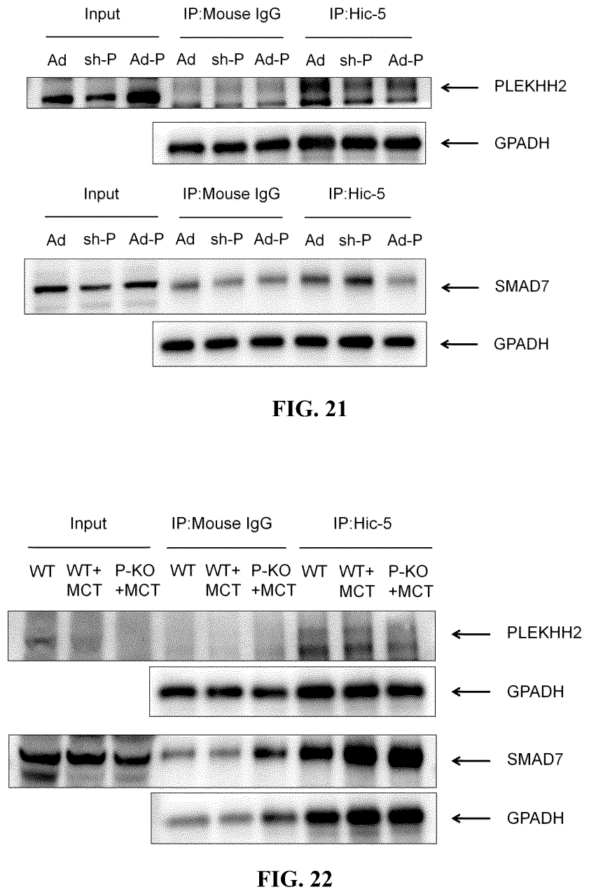

As shown in FIG. 21, the shRNA indeed knocks down the PLEKHH2 gene expression in the pulmonary vascular endothelial cells and also reduces the binding of the PLEKHH2 protein to Hic-5. At this point, the SMAD7 expression level does not significantly change, but the amount of binding to Hic-5 significantly increases. In contrast, after adenovirus overexpression of the PLEKHH2 gene, the amount of binding of PLEKHH2 to Hic-5 significantly increases, and the amount of binding of SMAD7 to Hic-5 significantly decreases.

The above cellular experiments clearly show that the binding of PLEKHH2 to Hic-5 is significantly negatively correlated with the binding of SMAD7 to Hic-5, indicating a clear competitive relationship between the two.

### Example 14: Competitive Binding of PLEKHH2 and SMAD7 to Hic-5 at the In Vivo Animal Level

To further clarify whether PLEKHH2 and SMAD7 competitively bind to Hic-5 in vivo, the PAH rat models were established using both subcutaneous injection of MCT and chronic hypoxia manners, respectively, and after successful model establishment, the lung tissues of the rats were collected to detect the binding of PLEKHH2 and SMAD7 to Hic-5 by the Co-IP experiment.

### (1) MCT Treatment Group

Three groups of rats were set up: 1) wild-type rats injected with normal saline as a control (WT); 2) wild-type rats at three weeks after injection of MCT (40 mg/kg) for (WT+MCT); and 3) PLEKHH2 gene heterozygous knockout rats at three weeks after injection of MCT (40 mg/kg) (P-KO+MCT). Total protein was extracted from the lung tissue to detect the levels of PLEKHH2 and SMAD7 proteins captured by the Hic-5 antibody.

Consistent with previous research findings of the examples in the present disclosure, the PLEKHH2 protein is significantly decreased in the lung tissue of the WT+MCT group rats and further decreased in the P-KO+MCT group rats. Correspondingly, the amount of the PLEKHH2 protein bound to Hic-5 is the highest in the WT group, is significantly reduced in the WT+MCT group, and is further reduced in the P-KO+MCT group. In contrast, the binding of the SMAD7 protein to Hic-5 is significantly increased in the WT+MCT group compared with the WT group and is further increased in the P-KO+MCT group (FIG. 22).

### (2) Chronic Hypoxia Group

Three groups of rats were set up: 1) wild-type rats (WT) housed under normoxia; 2) wild-type rats under chronic hypoxia (an oxygen concentration of 10% (v/v)) for three weeks (WT+Hypo); and 3) PLEKHH2 gene heterozygous knockout rats under chronic hypoxia (an oxygen concentration of 10% (v/v)) for three weeks (P-KO+Hypo). Total protein was extracted from the lung tissue to detect the levels of PLEKHH2 and SMAD7 proteins captured by the Hic-5 antibody.

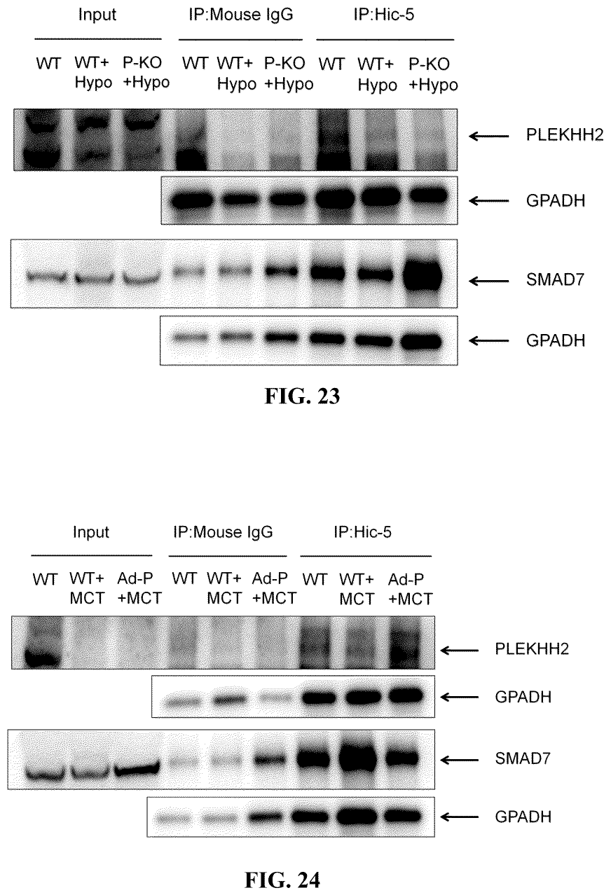

Consistent with the results of the MCT group described above, the level of the PLEKHH2 protein is significantly decreased in the lung tissue of the WT+Hypo group rats and further decreased in the P-KO+Hypo group rats. Correspondingly, the amount of the PLEKHH2 protein bound to Hic-5 is the highest in the WT group, is significantly reduced in the WT+Hypo group, and is further reduced in the P-KO+Hypo group. In contrast, the binding of the SMAD7 protein to Hic-5 is increased in the WT+Hypo group compared with the WT group, and is extremely significantly increased in the P-KO+Hypo group (FIG. 23).

Therefore, using the two animal models of PAH, it confirms that the binding of the two proteins, PLEKHH2 and SMAD7, to the Hic-5 protein in the lung tissue of the rats is significantly negatively correlated, and the clear competitive relationship exists between PLEKHH2 and SMAD7. Decreased PLEKHH2 expression promotes the binding of Hic-5 and SMAD7, and reduces the free SMAD7 protein. This molecular mechanism is consistent with our previous research findings that knockout of the PLEKHH2 gene exacerbates the phenotype of PAH animal models, suggesting that the imbalance among PLEKHH2, Hic-5, and SMAD7 is an important mechanism for the occurrence and development of PAH.

### Example 15: Binding of Hic-5 to SMAD7 Reduced by Increasing PLEKHH2 Expression in the Pathological Condition of PAH

Previous work has indicated that the reduced PLEKHH2 protein expression promotes the increased binding of Hic-5 to SMAD7. Therefore, it is investigated whether supplementation of PLEKHH2 could inhibit the binding of Hic-5 to SMAD7 in vivo. Two PAH rat models were established using MCT and Sugen+hypoxia, respectively. By intratracheal spray of the PLEKHH2 adenovirus, the expression of the PLEKHH2 gene was increased in lung tissues. After three weeks, the lung tissues of the rats were collected. The binding of PLEKHH2 and SMAD7 to Hic-5 was detected by the Co-IP experiment.

### (1) MCT Group Rats

The lung tissues were collected from three groups of rats: 1) wild-type rats injected with normal saline; 2) wild-type rats at 4 weeks after injection of MCT (50 mg/kg) (WT+MCT); and 3) on the day of MCT injection, the PLEKHH2 adenovirus was administered to the rats for a first time by intratracheal spray to upregulate the expression of the PLEKHH2 gene, the PLEKHH2 adenovirus was administered for a second time at 2 weeks, and the experiment was terminated at 4 weeks (Ad-P+MCT).

As shown in FIG. 24, the PLEKHH2 protein is significantly reduced in the WT+MCT group rats, and the binding of Hic-5 to SMAD7 is significantly enhanced. When the expression of the PLEKHH2 protein is significantly increased by the adenovirus, the binding level of Hic-5 to SMAD7 is reduced to a normal control level.

### (2) Sugen+Hypoxia Group Rats

The lung tissues were collected from three groups of rats: 1) wild-type rats injected with normal saline and housed under the normoxic condition for 5 weeks (WT); 2) a PAH rat model group (WT+SuHx) established by subcutaneously injecting Su-5416 (20 mg/kg) into the wild-type rats, followed by hypoxia (an oxygen concentration of 10% (v/v)) for 3 weeks and reoxygenation for 2 weeks; and 3) on the day of subcutaneous injection of Su-5416 into the rats, the PLEKHH2 adenovirus was administered to the rats for a first time by intratracheal spray to upregulate the expression of the PLEKHH2 gene, the PLEKHH2 adenovirus was administered for a second time at 3 weeks, and the experiment was terminated at 5 weeks (Ad-P+SuHx).

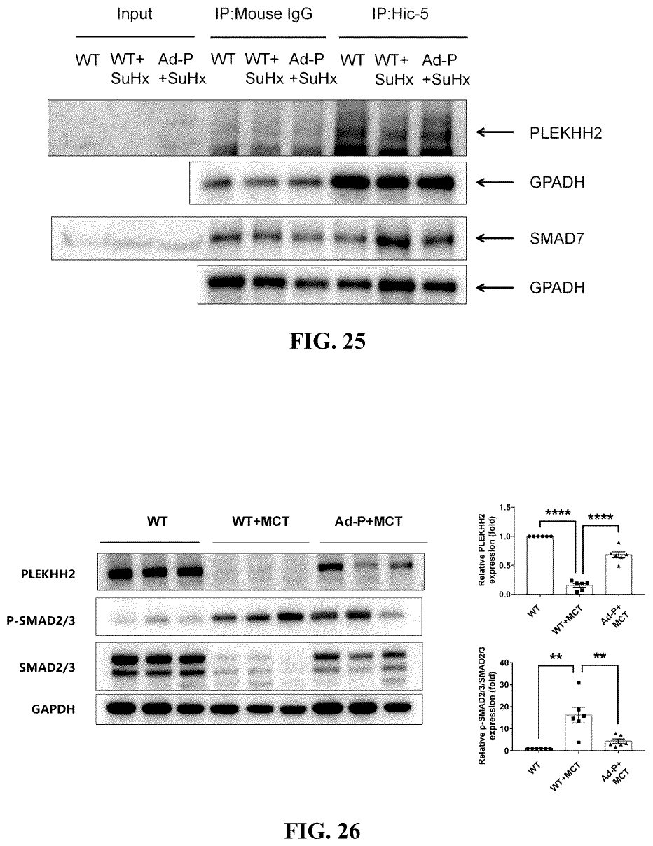

As shown in FIG. 25, the PLEKHH2 protein is significantly reduced in the WT+SuHx group rats, and the binding of Hic-5 to SMAD7 is significantly enhanced. When the expression of the PLEKHH2 protein is significantly increased by the adenovirus, the binding level of Hic-5 to SMAD7 is again reduced to near a normal control level.

Thus, it is confirmed that exogenous supplementation of the PLEKHH2 protein in the pathological condition of PAH significantly reduces the binding of Hic-5 to SMAD7 and increases free SMAD7 protein by two PAH animal models. This molecular mechanism is consistent with the previous research findings that supplementation of the PLEKHH2 gene can prevent or treat phenotypes of PAH animal models, suggesting that restoring the balance among PLEKHH2, Hic-5, and SMAD7 is an important strategy for treating or preventing PAH.

### Example 16: Prevention of PAH by PLEKHH2 Inhibiting the Hyperphosphorylation of SMAD2/3

As described above, SMAD7 is an important SMAD2/3 activity inhibitor. To confirm whether the SMAD7 protein released after PLEKHH2 competitively binds to Hic-5 is active, the lung tissues from the MCT group rats in the previous example was used to detect the PLEKHH2 protein expression and the SMAD2/3 phosphorylation. As shown in FIG. 26, after MCT stimulation, the PLEKHH2 protein is significantly reduced, the total protein of SMAD2/3 is significantly reduced, and the phosphorylated SMAD2/3 is significantly increased. After high expression of PLEKHH2 using the adenovirus, the total protein level of SMAD2/3 recovers to near the level of wild-type rats, and the phosphorylation level is significantly reduced.

The present disclosure first discovered that the PLEKHH2 and SMAD7 competitively bind to the Hic-5 protein in the lung tissues. In the pathological condition of PAH, the mutation or decreased expression of the PLEKHH2 protein leads to Hic-5 capturing more SMAD7, thereby preventing the inhibition of SMAD7 on SMAD2/3 activity, which in turn enhances the activity of the SMAD2/3 signaling pathway, promotes excessive proliferation of endothelial cells, accelerates EndoMT, and ultimately leads to PAH. Upregulating PLEKHH2 expression can prevent the binding of Hic-5 to SMAD7, allowing more SMAD7 to be released into the cytoplasm, inhibiting SMAD2/3 phosphorylation, thereby treating PAH.

Based on previous research findings, the molecular mechanism by which PLEKHH2 protects pulmonary vascular remodeling was further clarified, and a plurality of new approaches for treating PAH were proposed and verified.

(1) The results indicate that Hic-5 is highly expressed in the pathological condition of PAH, capturing more SMAD7 protein, thereby preventing the inhibition of SMAD7 on SMAD2/3. Therefore, degrading Hic-5 is a potential strategy for treating PAH.

(2) Designing small molecule compounds that mimic the binding site between Hic-5 and PLEKHH2 to reduce the binding of Hic-5 to SMAD7 represents a promising direction for new drug development.

Furthermore, it is experimentally confirmed that the aforementioned small molecule compounds that inhibit the binding of Hic-5 to SMAD7 can be effectively used in the treatment of PAH.

### Example 17: Hic-5 Directly Interacting with PLEKHH2 to Drive Pulmonary Vascular Remodeling as a New Potential Therapeutic Target

### 1. Experimental Materials

### 1.1 Databases and Software

R language (4.3.3), STRING database (Search Tool for the Retrieval of Interacting Genes, https://string-db.org/cgi/input.pl, 12.0), GeneCards database (THE HUMAN GENE DATABASE, https://www.genecards.org/), Alpha-Fold-3 molecular docking prediction software, and Pymol visualization software.

### 1.2 Cells and Reagents

TABLE 3
Cells

| Cell/Reagent Name | Catalog Number | Manufacturer |
|---|---|---|
| Human primary PAECs | HUM-iCell-a008 | Shanghai iCell |
| | | Bioscience Inc. |
| Human primary PMECs | HUM-iCell-a001 | Shanghai iCell |
| | | Bioscience Inc. |
| Human primary PASMCs | HUM-iCell-a009 | Shanghai iCell |
| | | Bioscience Inc. |
| DMEM/F12 medium | 10565018 | Thermo Fisher |
| | | Scientific, USA |

TABLE 4
Reagents

| | Catalog | |
|---|---|---|
| Reagent Name | Number | Manufacturer |
| 20 × Tris-buffered | T1080 | Beijing Solarbio Science & |
| saline (TBS) | | Technology Co., Ltd. |
| Protein A and Protein G | P2108 | Beyotime Biotechnology |
| (A + G) magnetic | | Co., Ltd. |
| beads | | |
| IP cell lysis buffer | P0013 | Beyotime Biotechnology |
| | | Co., Ltd. |
| Nuclear and cytoplasmic | P0028 | Beyotime Biotechnology |
| protein extraction kit | | Co., Ltd. |
| SMAD7 antibody | ab216428 | Abcam Trading Co., Ltd. |
| | | (Abcam) |
| SMAD7 antibody | sc-365846 | Santa Cruz Biotechnology |
| Poly(ADP-ribose) | | (Santa Cruz) |
| polymerase 1 (PARP-1) | sc-8007 | Santa Cruz Biotechnology |
| antibody | | (Santa Cruz) |
| Hic-5 antibody | 611164 | BD Biosciences, USA |
| Hic-5 antibody | PA5-17540 | Thermo Fisher Scientific, USA |
| Goat anti-rabbit IgG-647 | A-21245 | Thermo Fisher Scientific, USA |
| Goat anti-mouse IgG-647 | A-21235 | Thermo Fisher Scientific, USA |
| Goat anti-rabbit IgG-594 | A-11012 | Thermo Fisher Scientific, USA |
| Goat anti-mouse IgG-594 | A-11005 | Thermo Fisher Scientific, USA |

### 1.3 Experimental Animals

6-week-old male Sprague Dawley (SD) rats (Specific Pathogen-Free (SPF) grade), weighing 160-180 g, were purchased from Beijing HFK Bioscience Co., Ltd. and were housed in a clean and well-lit environment at a suitable temperature of (22±2° C.) and a humidity of (55±5) %, with free access to food and water.

### 2. Experimental Approaches

### 2.1 Cell Culture

(1) Preparation of complete cell culture medium. The complete cell culture medium for HPASMCs included Dulbecco's Modified Eagle Medium/Nutrient Mixture F-12 (DMEM/F12) basal medium supplemented, 10% Fetal Bovine Serum (FBS), and 1% Penicillin-Streptomycin (P/S). (2) Cell resuscitation. The cryopreserved cells were rapidly thawed in a 37° C. water bath. The thawed cell suspension was added to preheated culture medium and centrifuged at 800 rpm for 5 min to remove the supernatant. The cells were resuspended in fresh culture medium and transferred to a T-25 culture flask. (3) Cell subculture: a. the old culture medium was removed, the cells were washed once with sterile phosphate-buffered saline (PBS), and the PBS was removed; b. trypsin digestion: The HPASMCs were digested with 0.25% trypsin and incubated in an incubator for 2 min; c. cell digestion was observed, when the cells were approximately 80% digested, two volumes of the complete culture medium were added to stop the digestion; d. the cell suspension was transferred to a centrifuge tube and centrifuged at 800 rpm for 5 min, and the supernatant was removed; and e. the cells were resuspended in fresh culture medium and inoculated into new culture flasks at an appropriate density. (4) Cell plating: the cells were inoculated into 60 mm culture dishes, and the cell subculture protocol was performed up to the step of cell pellet resuspension. 20 μL of the cell suspension was taken and added to a cell counting chamber and counted in a cell counter. The cell stock solution was diluted according to a desired concentration. After mixing, the cell suspension was sequentially seeded dropwise by quadrant into culture dishes containing fresh culture medium.

### 2.2 PPI Network Construction

In the ‘Single Protein by Name/Identifier’ dialog box of the STRING database, the protein name ‘PLEKHH2’ was entered, and ‘Homo sapiens’ was selected for the species. After submission, the dataset generation conditions were adjusted in the settings tab: ‘evidence’ was selected for ‘Meaning of network edges’; ‘Textmining, Experiments, Databases, Co-expression, Neighborhood, Co-occurrence’ were checked for ‘Active interaction sources’; ‘medium confidence (0.400)’ was selected for ‘Minimum required interaction score’; and ‘no more than 10 interactors’ was selected for a 1st shell under ‘Max number of interactors to show’. A PPI network diagram was derived, with a PPI score greater than 0.4 considered statistically significant.

Additionally, PLEKHH2 was retrieved from the GeneCards database, and the PPI network diagram of PLEKHH2 was extracted.

### 2.3 Molecular Docking Prediction

The FERM domain of the PLEKHH2 protein and the Hic-5 full-length protein sequence, and the MH2 domain and N-terminal domain of the SMAD7 protein and the Hic-5 full-length protein sequence were respectively imported into the HDOCK software to perform protein interaction prediction. The Pymol software was used to visualize the docking results and to analyze the binding ability and binding site.

### 2.4 Cellular Immunofluorescence Staining

In the present experiment, primary antibodies against PLEKHH2 (with a dilution ratio of 1:200), Hic-5 (with a dilution ratio of 1:100), and SMAD7 (with a dilution ratio of 1:200) were employed, cells were incubated with the primary antibodies overnight at 4° C., and then recovered. Subsequently, the cells were washed with PBS for 5 minutes, repeated three times. Secondary antibodies (with a dilution ratio of 1:500) was incubated for 2 hours at the room temperature in the dark.

### 2.5 Co-IP

(1) Cell sample preparation: the culture medium was removed from the 60 mm culture dish. The cells were washed once with PBS, and residual liquid was aspirated. 100 μL of a lysis solution was added each dish, the lysis solution including IP lysis solution (Immunoprecipitation Lysis Buffer), Phenylmethylsulfonyl Fluoride (PMSF), and a protease phosphatase inhibitor. The cells were fully lysed by pipetting on ice. (2) Lung tissue sample preparation: a. on ice, 250 μL of a lysis solution was added to a grinding tube, and 50 mg of lung tissue was cut into pieces and placed into the tube. b. A frozen grinder was pre-cooled to 4° C. Program 1 was selected, including grinding M/S 21, a grinding time of 30 seconds, an interval time of 30 seconds, and 10 grinding cycles. After grinding, lysis was performed on ice for 30 minutes. (3) After lysis, the samples were centrifuged at 4° C. and 14000 g for 5-10 minutes, and the protein supernatant was transferred to a new 1.5 mL Eppendorf (EP) tube. (4) Sample and antibody incubation: An antibody or an IgG of the same species was added to the samples, such that the final concentration was 5-50 μg/mL, and the mixture was placed on the rotary mixer and incubated overnight at 4° C. (5) A+G magnetic bead preparation: a. washing: an appropriate amount of magnetic beads was transferred to a new 1.5 mL EP tube according to a ratio of 20 μL of magnetic bead suspension per 500 μL of sample. The tube was then placed on a magnetic rack for 10 seconds for separation, and the supernatant was discarded; b. 500 μL of 1×TBS was added. The magnetic beads were gently pipetted to resuspend. The tube was placed on the magnetic rack for 10 seconds for separation, and the supernatant was discarded. This step was repeated twice; c. resuspension: the magnetic beads were resuspended with 1×TBS to the initial volume. (6) Antibody complex and magnetic bead incubation: the resuspended magnetic beads were added to the sample-antibody complex in proportion, placed on the rotary mixer, and incubated at room temperature for 2 hours. (7) Magnetic separation: after the sample-antibody-magnetic bead incubation, the mixture was placed on the magnetic rack for 10 seconds for separation, and the supernatant was discarded. (8) Washing: 500 μL of 1×TBS was added to each tube. The magnetic beads were gently pipetted to resuspend. The tube was placed on the magnetic rack for 10 seconds for separation, and the supernatant was discarded. This step was repeated three times. (9) Elution and detection: a. elution solution: 100 μL of 1×Sodium Dodecyl Sulfate-Polyacrylamide Gel Electrophoresis (SDS-PAGE) loading buffer was added per 20 μL of magnetic beads; b. elution: the elution solution was added to each tube. The tubes were heated in a metal bath at 95° C. for 5 minutes. The tubes were placed on the magnetic rack for 10 seconds for separation, and the supernatant was collected for Western blot detection.

### 2.6 Construction of SuHx and MCT Rat Models

(1) After 1 week of acclimation feeding, twelve rats were randomly divided into two groups: a control group of 6 rats (WT) and a SuHx group of 6 rats (SuHx). The rats in the SuHx group were subcutaneously injected with 20 mg/kg SU5416 according to their body weight. They were then maintained in a hypoxia chamber for 3 weeks, and subsequently maintained in a normoxic environment for 2 weeks. The rats in the control group were injected with an equal volume of normal saline and maintained in a normoxic environment for 5 weeks. After 5 weeks, the model establishment was completed, and sampling was performed. (2) After 1 week of acclimation feeding, twelve rats were randomly divided into two groups: a control group of 6 rats (WT) and an MCT group of 6 rats (MCT). The rats in the MCT group were subcutaneously injected with 50 mg/kg MCT according to their body weight; and the rats in the control group were injected with an equal volume of normal saline. After 4 weeks, the model establishment was completed, and sampling was performed.

2.7 siRNA Transfection of Human Primary PASMCs

(1) The siRNA for Hic-5 was synthesized by Suzhou GenePharma Co., Ltd. (2) HPASMCs were seeded into a 60 mm cell culture dish and cultured until cell confluence reached 70%-80%. (3) A transfection mixture was prepared in advance: 100 μmol of siRNA and 9 μL of Lipofectamine 3000 reagent were added to 250 μL of DMEM culture medium, and gently mixed. (4) The old culture medium was aspirated. The cells were gently washed once with DMEM culture medium. The transfection mixture prepared in step (3) was gently dropped into the cell culture dish. After 10 minutes of incubation, 3 mL of DMEM culture medium containing 10% FBS was added. The dish was slowly shaken to ensure uniform distribution of the reagents. (5) After the culture dish was incubated at 37° C. for 6-8 hours, the medium was replaced with DMEM/F12 complete medium, and the cells were further cultured for 24 hours for subsequent experiments.

### 2.8 Verification of Hic-5 Knockdown Efficiency

The siRNA sequences targeting the Hic-5 gene were designed and synthesized, as shown in Table 4. The gene knockdown efficiency was verified through a Western blot experiment. The siRNA sequence with the highest knockdown efficiency was selected to perform Hic-5 gene knockdown on the HPASMCs, and cells treated with a negative control siRNA sequence were simultaneously used as a control group (Ctrl siR).

TABLE 4
Synthesized siRNA sequences

| Name | Species | Sequence (5′-3′) |
|---|---|---|
| Hic-5-siRNA | Homo sapiens | sense: GCAGCAGCUUCUUCGAGAATT (SEQ ID NO: 9) |
| (H-siR-1′) | | antisense: UUCUCGAAGAAGCUGCUGCTT (SEQ ID |
| | | NO: 10) |
| H-siR-2′ | Homo sapiens | sense: CCAUCCGACACAAGAUGGUTT (SEQ ID NO: 11) |
| | | antisense: ACCAUCUUGUGUCGGAUGGTT (SEQ ID NO: |
| | | 12) |
| H-siR-3′ | Homo sapiens | sense: CAUAAACACACGCAUUCCATT (SEQ ID NO: 13) |
| | | antisense: UGGAAUGCGUGUGUUUAUGTT (SEQ ID NO: |
| | | 14) |
| H-siR-4′ | Homo sapiens | sense: CGUUGUGCGAGAACCACUUTT (SEQ ID NO: 15) |
| | | antisense: AAGUGGUUCUCGCACAACGTT (SEQ ID NO: |
| | | 16) |
| H-siR-5′ | Homo sapiens | sense: GACAAGGACCACCUGUACATT (SEQ ID NO: 17) |
| | | antisense: UGUACAGGUGGUCCUUGUCTT (SEQ ID NO: |
| | | 18) |
| Ctrl-siR | Homo sapiens | sense: UUCUCCGAACGUGUCACGUTT (SEQ ID NO: 19) |
| | | antisense: ACGUGACACGUUCGGAGAATT (SEQ ID NO: |
| | | 20) |

### 2.9 Cell Grouping and Treatment

The HPASMCs to be transfected were seeded into 60 mm cell culture dishes, and a negative control sequence (Ctrl-siR) and an siRNA sequence (H-siR-1′) for knocking down Hic-5 were respectively added to each culture dish. Subsequently, the cells were processed according to the operations described in the siRNA transfection experiment.

### 2.10 Edu Assay in Human Primary PASMCs

(1) Platelet-Derived Growth Factor-BB (PDGF-BB) treatment group: The HPASMCs from each transfected group were seeded into 24-well plates at a density of 3×10⁴ cells/well, and after culturing in a 37° C. cell incubator for 24 h, the DMEM-F12 basal medium containing PDGF-BB was added. The concentration of the PDGF-BB was 20 ng/ml, and the treatment time was 24 h. (2) Non-PDGF-BB treatment group: The HPASMCs from each transfected group were seeded into the 24-well plates at a density of 3×10⁴ cells/well, and after culturing in a 37° C. cell incubator for 24 h, the same volume of the DMEM-F12 basal medium as that of the PDGF-BB treatment group was added. (3) Edu Labeling: 1 mL of a 1× Edu working solution (10 μM) prepared with the DMEM-F12 complete medium was added to each well, and the wells were continued to be incubated for 6 to 8 hours. (4) Fixation: The culture medium was discarded, and 4% (w/v) paraformaldehyde was added to fix the cells for 15 minutes. (5) Washing: The fixing solution was aspirated, and the cells were washed twice with a washing solution (PBS containing 3% (w/v) bovine serum albumin (BSA)), with 3 to 5 minutes each time. (6) Permeabilization: A permeabilization solution (PBS containing 0.3% (w/v) TritonX-100) was added and incubated at room temperature for 15 minutes. (7) Re-washing: The permeabilization solution was aspirated, and the cells were washed once with the washing solution. (8) Edu reaction staining: 200 μL of an Edu reaction mixture was added to each well, and incubated on a shaker in the dark for 30 minutes; the preparation ratio of the Edu reaction mixture was as follows: a total volume of 2.5 mL=2.15 mL 1× Click reaction buffer+100 μL CuSO4+5 μL 488-azide+250 μL 1× Click reaction buffer additive. (9) Nuclear staining: The Edu reaction mixture was aspirated, and after washing twice with the washing solution, 200 μL of a Hoechst 33342 dye solution (500×) was added to each well, and after incubation at room temperature in the dark for 10 minutes, the dye solution was aspirated, and the cells were washed twice with the washing solution. (10) High-content imaging fluorescence detection.

### 2.11 Wound Healing Assay of Human Primary PASMCs

(1) PDGF-BB treatment group: The HPASMCs from each transfected group were seeded into the 96-well plates at a density of 6000 cells/well, and after culturing in a 37° C. cell incubator for 24 hours, a blank medium containing PDGF-BB was added. The concentration of the PDGF-BB was 20 ng/mL, and the treatment time was 24 hours. (2) Non-PDGF-BB treatment group: The HPASMCs from each transfected group were seeded into the 96-well plates at a density of 6000 cells/well, and after culturing in a 37° C. cell incubator for 24 hours, the same volume of the DMEM-F12 basal medium as that of the PDGF-BB treatment group was added. (3) Wound scratching: A scratcher was pre-placed into a biological safety cabinet for ultraviolet (UV) sterilization, and after the sterilization was completed, the 96-well plates were placed into the scratcher for scratching. After the scratching was completed, the complete medium from the cells was aspirated, and 200 μL of the DMEM-F12 basal medium containing PDGF-BB was respectively added to each well. (4) Continuous photography was performed using an Incucyte imaging system.

### 2.12 Detection of Cell Apoptosis Using Caspase3/7 Kit

(1) PDGF-BB treatment group: The HPASMCs from each transfected group were seeded into the 96-well plates at a density of 6000 cells/well, and after culturing in a 37° C. cell incubator for 24 hours, a blank medium containing PDGF-BB was added. The concentration of PDGF-BB was 20 ng/ml, and the treatment time was 24 hours. (2) Non-PDGF-BB treatment group: HPASMCs from each transfected group were seeded into the 96-well plate at the density of 6000 cells/well, and after culturing in the 37° C. cell incubator for 24 hours, the same volume of the DMEM/F12 basal medium as that of the PDGF-BB treatment group was added. (3) Preparation: After equilibrating Caspase-Glo 3/7 buffer and lyophilized Caspase-Glo 3/7 substrate at room temperature, the buffer was added to an amber bottle containing the substrate, and mixed by rotation or inversion until the substrate was completely dissolved. (4) Collection of cell supernatant: 100 μL of cell supernatant of each group was collected separately and added to a white 96-well plate. (5) Addition of samples: 100 μL of the Caspase-Glo3/7 mixture was added to each sample well, and the 96-well plate was covered with a sealing film. (6) Incubation: After gently mixing the contents in the 96-well plate using a plate shaker, the plate was placed in a 25° C. constant temperature incubator for 1 hour of incubation. (7) Reading: In a microplate reader, the 96-well white plate was selected, a ‘Luminescence’ mode was chosen, a reading time was set to ‘1 sec’, and a chemiluminescence value was read.

### 3. Experimental Results

### 3.1 Interaction Between PLEKHH2 and TGFβ1I1 (Hic-5) Suggested by PPI Analysis

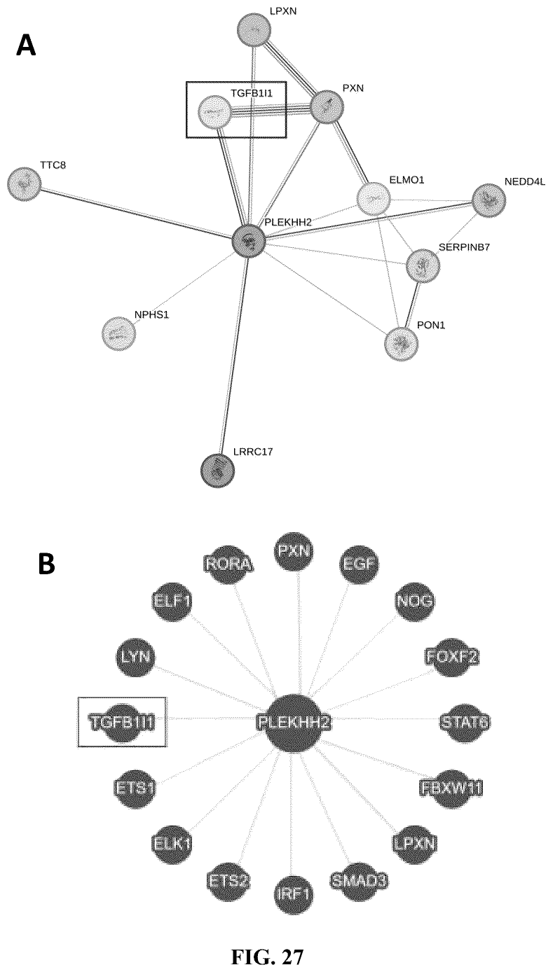

A PPI network diagram (FIG. 27) with PLEKHH2 as the core was constructed using the STRING and GeneCards databases, separately. The analysis results show that both databases include the upstream regulatory factor TGFβ111 (Hic-5) of the TGF-β signaling pathway, suggesting that PLEKHH2 can regulate the TGF-β signaling pathway through the interaction with TGFβ1I1 (Hic-5).

3.2 Prediction of PLEKHH2 Competitively Binding to the LIM3 Domain of Hic-5 Protein with SMAD7 by Molecular Docking

Literature reports demonstrate that the FERM domain of PLEKHH2 protein binds to the Hic-5 protein, while the N-terminal domain and the MH2 domain of SMAD7 protein directly bind to the LIM3 domain of Hic-5. However, the specific binding region between the PLEKHH2 protein and the Hic-5 protein is not clear.

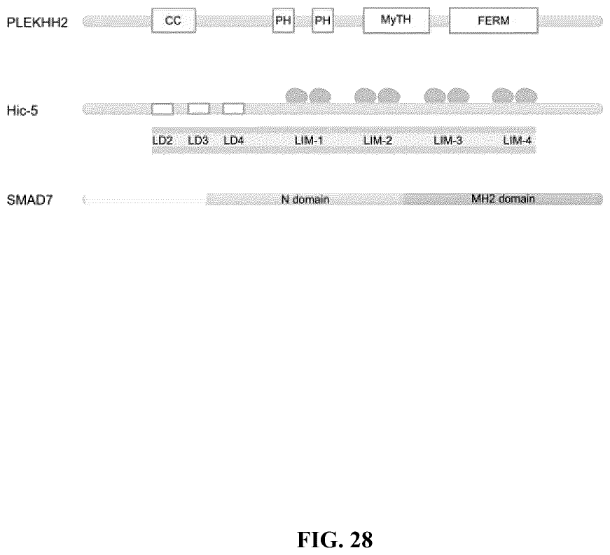

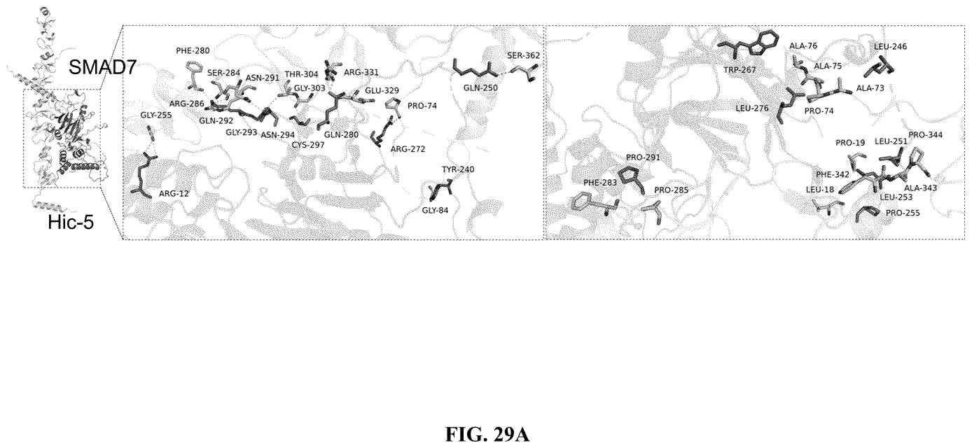

To this end, a schematic diagram of protein structures of PLEKHH2, Hic-5, and SMAD7 was first drawn (FIG. 28). PLEKHH2 protein is composed of five domains, including a CC domain, two PH domains, a MyTH domain, and a FERM domain. Hic-5 protein contains seven main functional domains, including three LD domains and four LIM domains. SMAD7 protein is composed of an N-terminal domain and an MH2 domain.

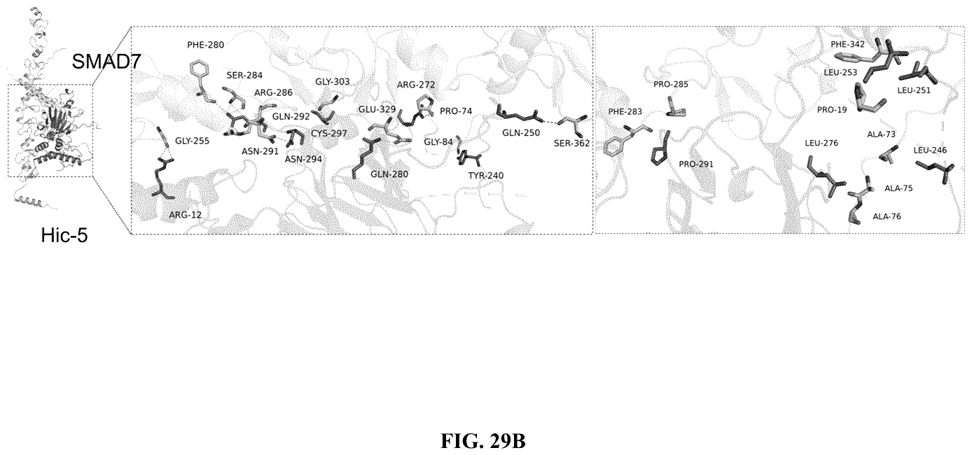

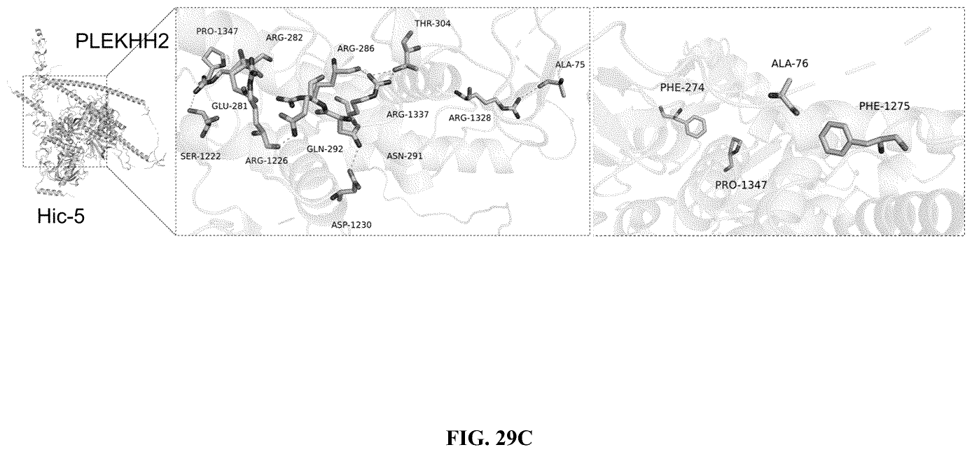

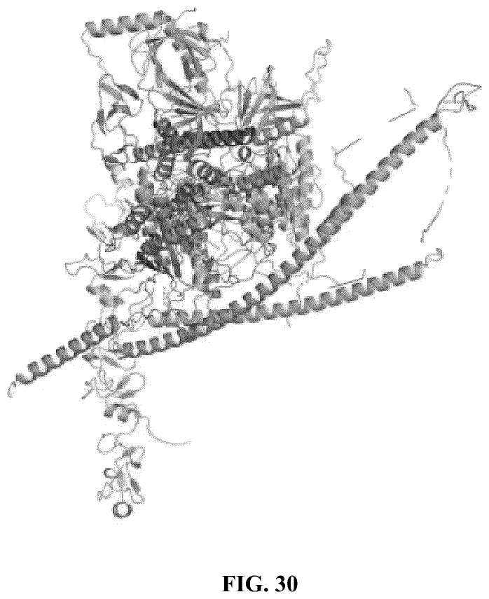

Subsequently, the molecular docking was performed using HDOCK software, and a visualization analysis was conducted using Pymol software to study the binding ability and binding regions among PLEKHH2, Hic-5, and SMAD7 proteins. The results (FIGS. 29A-29C) show that the docking score of the N-terminal domain of SMAD7 with the LIM3 domain of Hic-5 protein is −260.01, the docking score of the MH2 domain of SMAD7 with the LIM3 domain of Hic-5 protein is −247.48, and the docking score of the FERM domain of PLEKHH2 with the LIM3 domain of Hic-5 is −278.67, all three of which primarily rely on hydrogen bonds and hydrophobic interactions. The analysis (FIG. 30) reveals that the binding region between the N-terminal domain of SMAD7 and Hic-5 overlaps with the binding region between the FERM domain of PLEKHH2 and the LIM3 domain of Hic-5.

In summary, PLEKHH2 and SMAD7 can competitively bind to the LIM3 domain of Hic-5, suggesting a competitive relationship between them when binding to Hic-5.

### 3.3 Colocalization of PLEKHH2 and Hic-5 in HPAECs

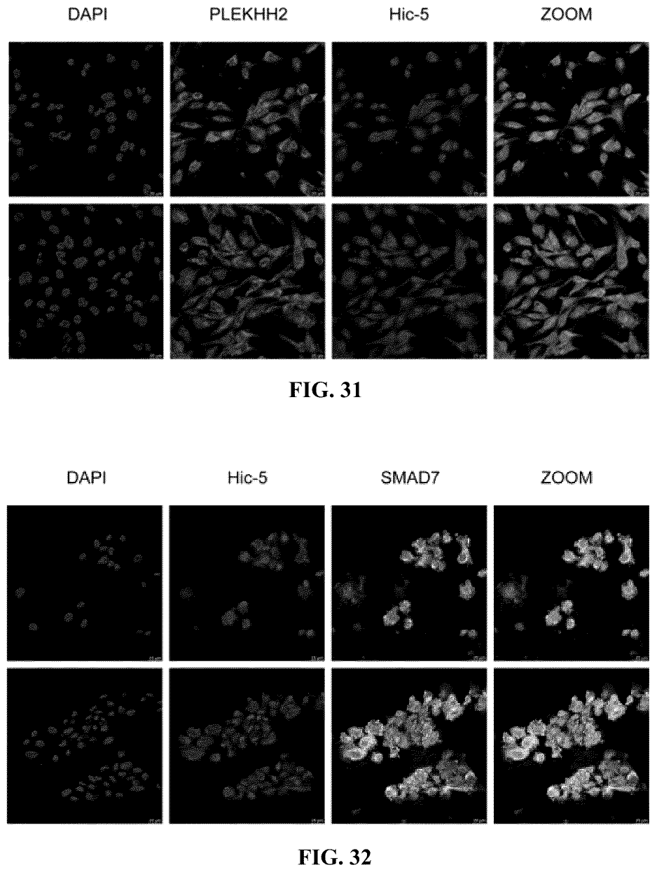

In the cultured HPAECs in vitro, the expression of PLEKHH2 and Hic-5 was confirmed by the immunofluorescence experiment, and it was found that their localization in the HPAECs was highly overlapping (FIG. 31).

### 3.4 Colocalization of Hic-5 and SMAD7 in HPAECs

In the cultured HPAECs in vitro, the immunofluorescence experiment found that both Hic-5 and SMAD7 were expressed in the HPAECs, and their localization was highly overlapping (FIG. 32).

3.5 Validation of PLEKHH2 Competitively Binding to the Hic-5 Protein with SMAD7 at the Cellular Level

To verify whether PLEKHH2 competitively binds to Hic-5 with SMAD7, in the cultured HPAECs in vitro, the PLEKHH2 expression was knocked down using the shRNA (sh-P), and PLEKHH2 was overexpressed using the adenovirus (Ad-P). Subsequently, the effect of changes in PLEKHH2 expression on the binding of Hic-5 and SMAD7 was detected through the Co-IP experiment.

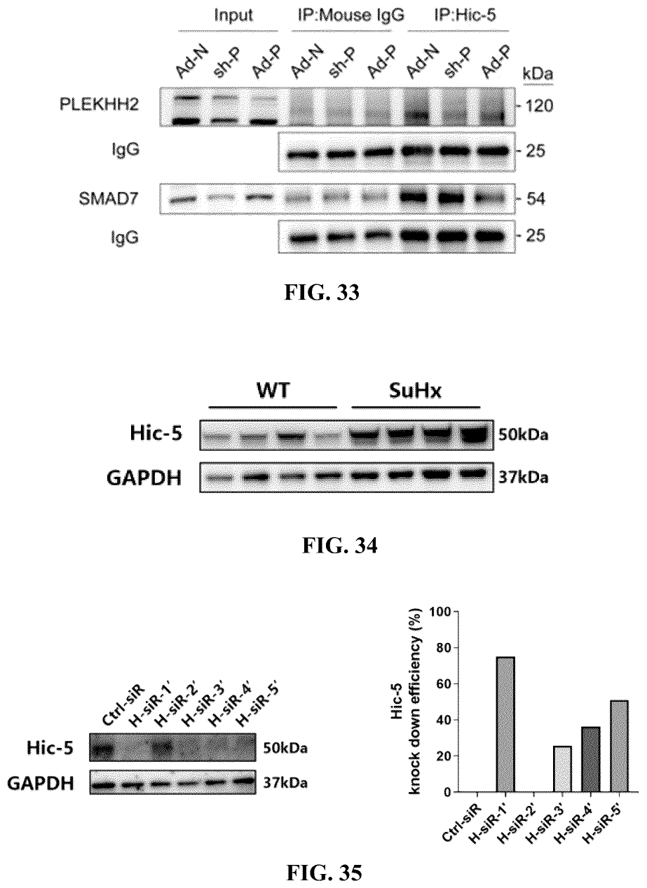

First, after performing the Co-IP using magnetic beads coated with the Hic-5 antibody, the Western blot analysis showed that the PLEKHH2 protein was enriched (FIG. 33). Meanwhile, the detection using the SMAD7 antibody also found that the SMAD7 protein was enriched (FIG. 33), indicating that Hic-5 can bind to PLEKHH2 and SMAD7 separately.

Next, the effect of changes in PLEKHH2 expression on the protein binding was analyzed. After the PLEKHH2 knockdown with shRNA, the expression of PLEKHH2 in HPAECs decreased, and the amount of PLEKHH2 protein bound to Hic-5 also decreased. At this time, the expression of SMAD7 protein decreased, while the amount of SMAD7 protein bound to Hic-5 increased. In contrast, when the adenovirus overexpressed PLEKHH2, the expression of PLEKHH2 protein increased, and the amount of PLEKHH2 protein bound to Hic-5 also increased accordingly, while the expression of SMAD7 protein increased, and the amount of SMAD7 protein bound to Hic-5 decreased.

The above cellular level experiments indicate that there is a negative correlation between the binding of PLEKHH2 to Hic-5 protein, and the blinding of SMAD7 to Hic-5 protein, suggesting the existence of a competitive binding mechanism between the two.

### 3.6 Increased Expression of Hic-5 in Lung Tissue of PAH Model Rats

The expression level of Hic-5 in the lung tissue of SuHx model rats was detected by the Western blot analysis. Compared with WT rats, the Hic-5 expression level in the lung tissue of the SuHx rats increases by about 75% (FIG. 34), suggesting that Hic-5 can play a key regulatory role in the progression of PAH disease.

3.7 Decreased Expression of the Hic-5 in HPASMCs by siRNA Transfection

After transfecting HPASMCs with five different siRNAs, the Western blot experimental results show that H-siR-1′, as the siRNA fragment with the highest knockdown efficiency in the batch, achieves the knockdown efficiency of up to 75.12% (FIG. 35). Therefore, in subsequent experiments, H-siR-1′ was chosen for the transfection to knock down the Hic-5 expression.

### 3.8 Inhibition of PDGF-BB-Induced Abnormal Proliferation of HPASMCs by Hic-5 Knockdown

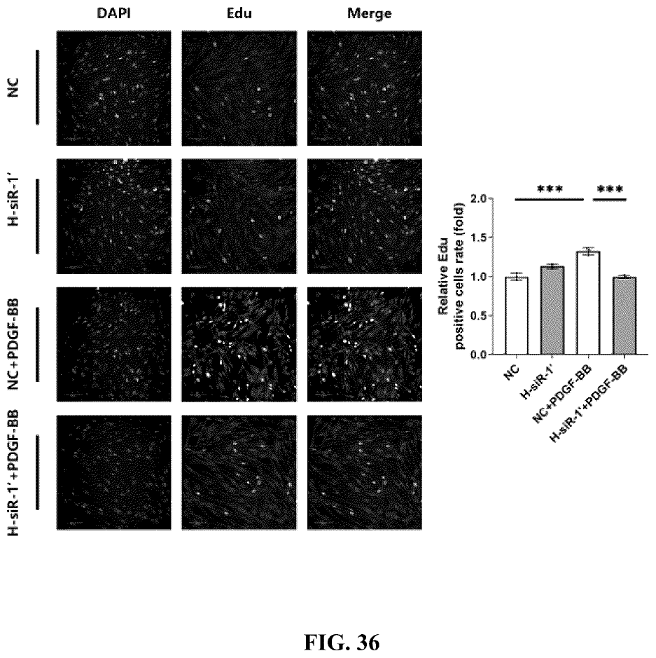

The DNA replication of HPASMCs was assessed by the Edu assay to reflect cell proliferation ability. As shown in FIG. 36, in the PDGF-BB induced HPASMCs, the Edu positive rate increases by about 32.5% compared with the negative control (NC) group, suggesting that the DNA replication is increased and the proliferation ability is enhanced. While in the HPASMCs with Hic-5 knockdown by H-siR-1′ after the PDGF-BB induction, the Edu positive rate decreases by about 24.7% compared with the NC-PDGF-BB group, suggesting the cell DNA replication is reduced and the proliferative effect of PDGF-BB is significantly inhibited.

### 3.9 Increased HPASMCs Apoptosis by Hic-5 Knockdown

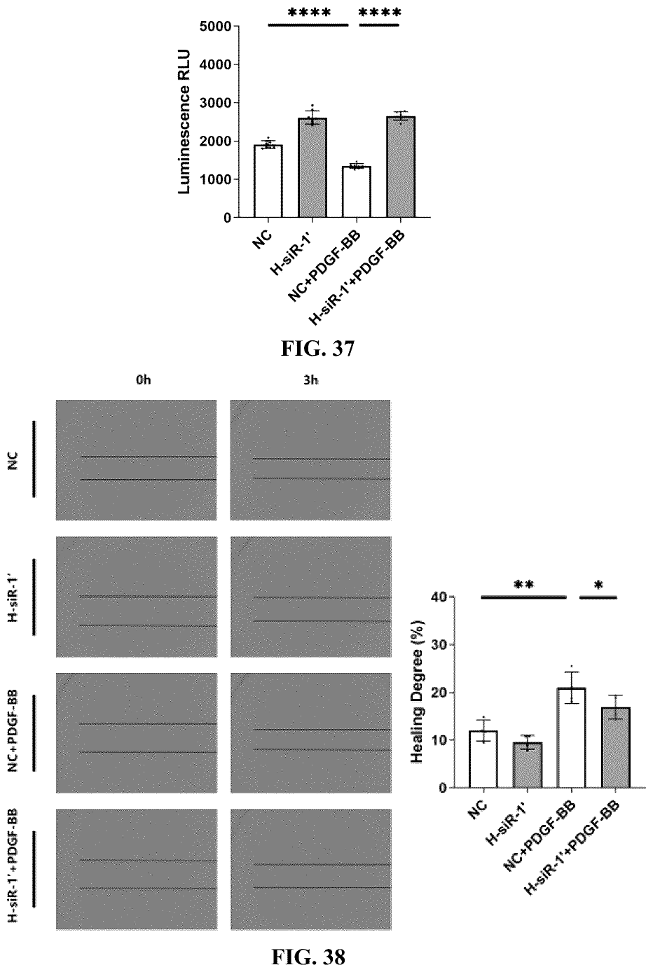

The Caspase3/7 kit was used to detect the Caspase3/7 activity in the cell supernatant to reflect the cell apoptosis status. As shown in FIG. 37, the PDGF-BB induction reduces the HPASMCs apoptosis rate by about 29.2%, while the Hic-5 knockdown reverses the anti-apoptotic effect of PDGF-BB.

### 3.10 Inhibition of HPASMCs Migration by Hic-5 Knockdown

The effect of the Hic-5 knockdown on the migration ability of HPASMCs was detected by a wound healing assay. As shown in FIG. 38, the healing rate of the PDGF-BB treatment group is about 74.8% higher than that of the control group, and the Hic-5 knockdown reduces the pro-migratory ability of PDGF-BB by about 19.4%.

### 3.11 Effectively Prevention of PAH Induced by MCT and Hypoxia+Sugen by In Vivo Hic-5 Knockdown

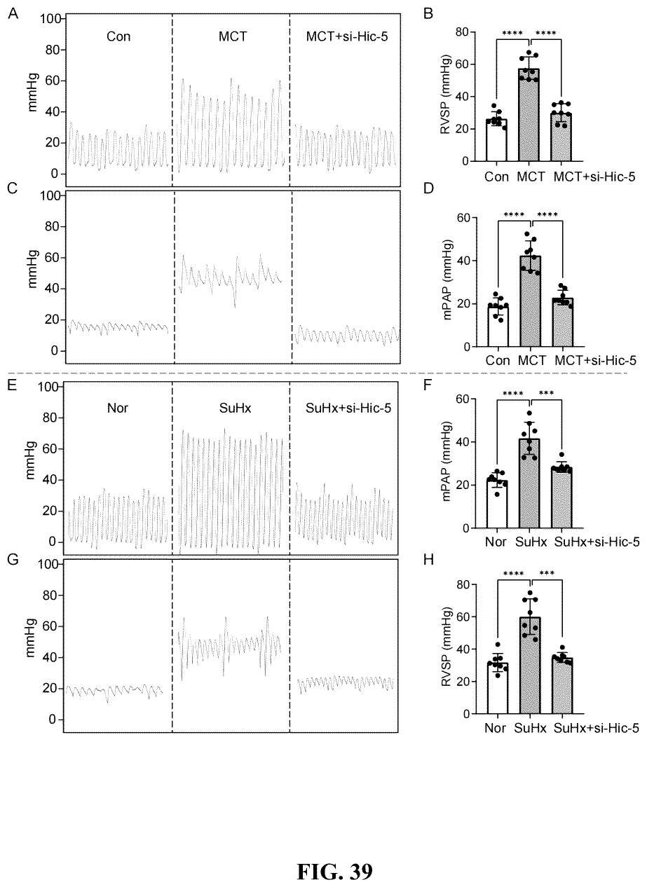

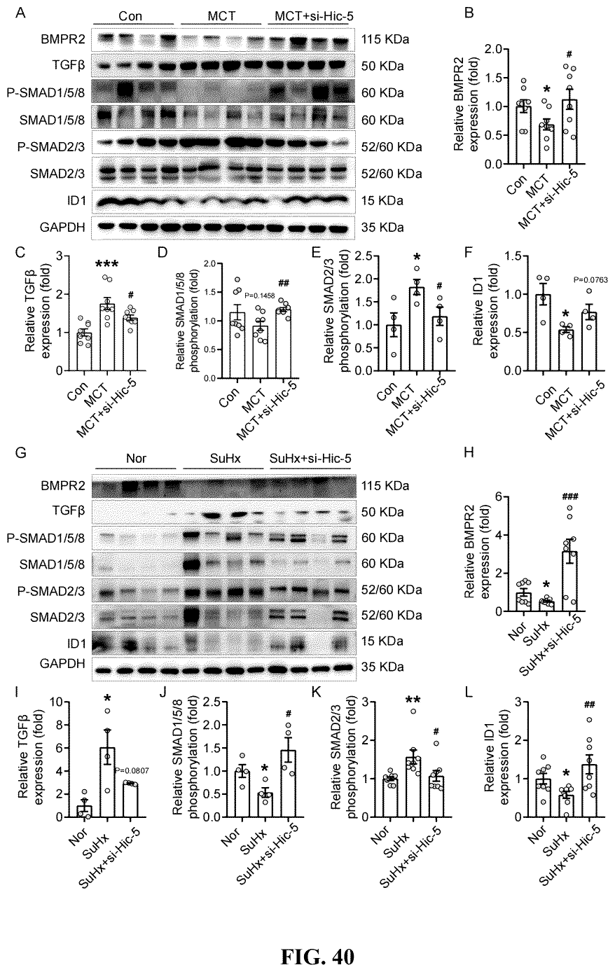

Through the above studies, Hic-5 is identified to be a core downstream molecule for PLEKHH2 to exert its pulmonary vascular protective effect. At the in vitro level, Hic-5 may be a detrimental gene′ that promotes pulmonary vascular remodeling, and the knockdown of Hic-5 is likely to protect against pulmonary vascular remodeling. A siRNA targeting Hic-5 (si-Hic-5) adenovirus vector was constructed and Hic-5 was knocked down in pulmonary vessels through tracheal atomization. As shown in FIG. 39, the results indicate that the inhibition of Hic-5 significantly reduces the PAH induced by MCT and hypoxia+Sugen. The signal pathway detection shows that after Hic-5 knock down, the BMPR2 pathway activity increases, the TGF-β pathway activity decreases, and the BMPR2-TGF-β pathway, which had previously lost balance under the pathological condition of PAH, is re-balanced (FIG. 40), achieving a good effect of preventing the PAH.

The description of the above embodiments is merely for understanding the methods and its core idea of the present disclosure. It is to be understood that, for a person of ordinary skill in the art, a plurality of improvements and modifications may also be made to the present disclosure without departing from the principles of the present disclosure, and these improvements and modifications will also fall within the scope of the claims.

What is claimed is:

1. A method for prevention and/or treatment of pulmonary arterial hypertension, comprising: administering to a subject at least one of a first agent that inhibits binding of Hic-5 to SMAD7 or a second agent that inhibits Hic-5.

2. The method according to claim 1, wherein the first agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting a binding site between Hic-5 and SMAD7; and
the second agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting Hic-5.

3. The method according to claim 1, wherein the binding site between Hic-5 and SMAD7 is a LIM3 domain of Hic-5.

4. The method according to claim 1, wherein the first agent competitively binds to a LIM3 domain of Hic-5 with SMAD7.

5. The method according to claim 1, wherein the first agent comprises at least one of PLEKHH2 or a PLEKHH2 activator.

6. The method according to claim 5, wherein the at least one of PLEKHH2 or the PLEKHH2 activator comprises at least one of a PLEKHH2 gene, a PLEKHH2 mRNA, a PLEKHH2 cDNA, a PLEKHH2 protein, a peptide fragment of the PLEKHH2 protein, an analog of the PLEKHH2 protein, an active fragment of any of the foregoing, a vector expressing PLEKHH2, nanoparticles carrying the PLEKHH2 gene, a viral vector carrying the PLEKHH2 gene, a PEGylated protein encapsulating the PLEKHH2 gene or the PLEKHH2 protein, protein microspheres encapsulating the PLEKHH2 gene or the PLEKHH2 protein, a liposome encapsulating the PLEKHH2 gene or the PLEKHH2 protein, or extracellular vesicles encapsulating the PLEKHH2 gene or the PLEKHH2 protein.

7. The method according to claim 5, wherein the at least one of PLEKHH2 or the PLEKHH2 activator prevents, treats, alleviates, and/or ameliorates the pulmonary arterial hypertension by inhibiting hyperphosphorylation of SMAD2/3.

8. The method according to claim 1, wherein the second agent is selected from a group comprising an siRNA targeting a Hic-5 gene, an shRNA targeting the Hic-5 gene, an agent that inhibits transcriptional activity of the Hic-5 gene, an agent that inhibits a transcription level of an Hic-5 mRNA, an agent that promotes degradation of the Hic-5 mRNA, an agent that inhibits translation of the Hic-5 mRNA, an agent that specifically recognizes the Hic-5 gene with a guide nucleic acid and cleaves the Hic-5 gene to reduce an expression level of Hic-5, or a reagent for partial or complete knockout of the Hic-5 gene.

9. The method according to claim 8, wherein the siRNA targeting the Hic-5 gene has a sequence shown in SEQ ID NOs: 9-10.

10. The method according to claim 8, wherein the second agent is capable of reducing the pulmonary arterial hypertension, increasing BMPR2 pathway activity, reducing TGF-β pathway activity, or restoring BMPR2-TGF-β pathway balance.

11. A method for prevention and/or treatment of pulmonary arterial hypertension, comprising:
administering to a subject at least one of PLEKHH2 or a PLEKHH2 activator.

12. A pharmaceutical composition for prevention and/or treatment of pulmonary arterial hypertension, comprising at least one of a first agent that inhibits binding of Hic-5 to SMAD7 or a second agent that inhibits Hic-5; wherein
the first agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting a binding site between Hic-5 and SMAD7; and
the second agent comprises at least one of a small molecule compound, a polypeptide, a peptidomimetic, a nucleic acid molecule, a protein, an antibody, an antibody mimetic, a fusion protein, a protein analog, a gene delivery vector, a protein delivery vector, an aptamer, an shRNA, an siRNA, an miRNA, an antisense nucleic acid, or a CRISPR gene editing agent targeting Hic-5.

13. The pharmaceutical composition according to claim 12, further comprising other drugs for prevention and/or treatment of the pulmonary arterial hypertension.

14. The pharmaceutical composition according to claim 13, wherein the other drugs for prevention and/or treatment of the pulmonary arterial hypertension comprise at least one of a calcium channel blocker, a prostacyclin analog, an endothelin receptor antagonist, a phosphodiesterase-5 inhibitor, or a guanylate cyclase stimulator.

15. The pharmaceutical composition according to claim 12, further comprising at least one of a pharmaceutically acceptable carrier or an excipient.

16. A pharmaceutical preparation for prevention and/or treatment of pulmonary arterial hypertension, comprising the pharmaceutical composition of claim 12.

17. The pharmaceutical preparation according to claim 16, wherein a dosage form of the pharmaceutical preparation comprises at least one of an injection, a lyophilized powder, a solution, a tablet, a capsule, a granule, an ointment, a cream, a gel, a suspension, an oral solution, a controlled-release formulation, a nanocrystal formulation, a microemulsion, or a solid dispersion.

18. A method for screening a candidate drug for prevention and/or treatment of pulmonary arterial hypertension, comprising:
screening an agent that binds to a LIM3 domain of Hic-5 as the candidate drug; or
simulating a binding site between Hic-5 and PLEKHH2, and screening an agent that binds to the binding site between Hic-5 and PLEKHH2 as the candidate drug; or
simulating a binding site between Hic-5 and SMAD7, and screening an agent that binds to the binding site between Hic-5 and SMAD7 as the candidate drug; or
treating a system expressing or containing a PLEKHH2 gene with a test substance; detecting an expression level of the PLEKHH2 gene in the system; and selecting a test substance that increases the expression level of the PLEKHH2 gene as the candidate drug.

19. A method for treating pulmonary arterial hypertension, comprising:
detecting an expression level of PLEKHH2 in a sample from a subject; and
comparing the sample from the subject with a control sample and administering an effective amount of at least one of PLEKHH2 or a PLEKHH2 activator to the subject in response to determining that the expression level of PLEKHH2 in the sample from the subject is lower than that in the control sample.

20. The method according to claim 19, wherein the sample from the subject comprises at least one of blood, tissues, blood-derived cells, serum, plasma, lymph, synovial fluid, or cells from the subject.


SEQUENCE LISTING (20 sequences)

SEQ ID NO: 1 - DNA, 18 bp, synthetic construct
  source 1..18: mol_type = other DNA
agcggacgactcaagact

SEQ ID NO: 2 - DNA, 18 bp, synthetic construct
  source 1..18: mol_type = other DNA
tgaaccgtttgcttgtta

SEQ ID NO: 3 - DNA, 20 bp, synthetic construct
  source 1..20: mol_type = other DNA
ctcatgaccacagtccatgc

SEQ ID NO: 4 - DNA, 20 bp, synthetic construct
  source 1..20: mol_type = other DNA
cacattgggggtaggaacac

SEQ ID NO: 5 - DNA, 21 bp, synthetic construct
  source 1..21: mol_type = other DNA
gaggaaatgagcaagatattt

SEQ ID NO: 6 - DNA, 21 bp, synthetic construct
  source 1..21: mol_type = other DNA
atatcttgctcatttcctctt

SEQ ID NO: 7 - DNA, 21 bp, synthetic construct
  source 1..21: mol_type = other DNA
ggcttctgaaagtgattattt

SEQ ID NO: 8 - DNA, 21 bp, synthetic construct
  source 1..21: mol_type = other DNA
ataatcactttcagaagcctt

SEQ ID NO: 9 - RNA, 21 nt, synthetic construct
  source 1..21: mol_type = other RNA
gcagcagcttcttcgagaatt

SEQ ID NO: 10 - RNA, 21 nt, synthetic construct
  source 1..21: mol_type = other RNA
ttctcgaagaagctgctgctt

SEQ ID NO: 11 - RNA, 21 nt, synthetic construct
  source 1..21: mol_type = other RNA
ccatccgacacaagatggttt

SEQ ID NO: 12 - RNA, 21 nt, synthetic construct
  source 1..21: mol_type = other RNA
accatcttgtgtcggatggtt

SEQ ID NO: 13 - RNA, 21 nt, synthetic construct
  source 1..21: mol_type = other RNA
cataaacacacgcattccatt

SEQ ID NO: 14 - RNA, 21 nt, synthetic construct
  source 1..21: mol_type = other RNA
tggaatgcgtgtgtttatgtt

SEQ ID NO: 15 - RNA, 21 nt, synthetic construct
  source 1..21: mol_type = other RNA
cgttgtgcgagaaccactttt

SEQ ID NO: 16 - RNA, 21 nt, synthetic construct
  source 1..21: mol_type = other RNA
aagtggttctcgcacaacgtt

SEQ ID NO: 17 - RNA, 21 nt, synthetic construct
  source 1..21: mol_type = other RNA
gacaaggaccacctgtacatt

SEQ ID NO: 18 - RNA, 21 nt, synthetic construct
  source 1..21: mol_type = other RNA
tgtacaggtggtccttgtctt

SEQ ID NO: 19 - RNA, 21 nt, synthetic construct
  source 1..21: mol_type = other RNA
ttctccgaacgtgtcacgttt

SEQ ID NO: 20 - RNA, 21 nt, synthetic construct
  source 1..21: mol_type = other RNA
acgtgacacgttcggagaatt